SEQUENCE LISTING (22 sequences)

SEQ ID NO: 1 - DNA, 1709 bp, Salmonella typhimurium
aacagatgatgagaaagcgcggctcgacgaatggaaaaaatatagggtgctggtaaaccggatggatacagccgcccctgactggccggaaagaccagccagccagtaggcgttactgtggtaatgcaggccacctgattgctgtgaaggtggcctcatctgaaacacctgttaagtccagcgatttaagaacattgatataattcatcggcagaatcaaagctgctttattttcatcactgataatccccagcgttaattctatgcgccagtccttaatggcattatctgaatcgttaagtaatttctgccgggtgacttctgcctgccaaaattgaacgccgggcgttgataccggatgatctggtcatcgtggaaagcgaccctgaaaaaatcgacacttagctgtaaaatgacagtcccgccatccggtcatcataacggatttttcttctgcaccttctgaagcccgccatggcaggacgaccatgaatcccgccgataaccttattgtgaaattaagaccaggaagagatgatgtctgtcggacagatactatatgtaaatttataaaggttttttgttatgccctttcatattggaagcggatgtcttcccgccatcatcagtaaccgccgcatttatcgtattgcctggtctgataccccccctgaaatgagttcctgggaaaaaatgaaggaatttttttgctcaacgcaccaggctgaagcgctggagtgcatctggacgatttgtcacccgccggccggaacgacgcgggaggatgtggtcagcagatttgaactgctcaggacgctcgcgtatgacggatgggaggaaaacattcattccggcctgcacggggaaaactacttctgtattctggatgaaggcagtcaggagatattatcagtcaccctggatgacgtcgggaactataccgtaaattgccaggggtacagtgaaacacatcacttaaccatggcaacagaaccgggagtggaacgcacagatataacttacaacctaaccagtgatattgatgctgcggcctatctggaggaattgaaacagaatccaattataaataataaaataatgaatccggtagggcagtgtgagtcattaatgactcctgtaagcaattttatgaatgaaaaagggttcgataatattcgttatcgaggtatatttatctgggataaaccaacagaggaaataccaacaaatcattttgcagtggttggaaataaagaaggaaaagactatgtgtttgatgtttcagcccatcagtttgaaaatagaggtatgagtaatctgaatggcccattaattctttcagcagatgaatgggtttgtaaatatagaatggcaacaagaaggaaacttatttattatactgattttagtaattcaagtatagcagctaatgcctatgatgcattaccacgagaattagaatcagaatctatggcagggaaagtttttgttacatcaccgagatggtttaatacctttaaaaagcaaaaatattccttaataggtaaaatgtaagcgcaccgtggaggacgtctgtcagaaccctgtcaatccggcgatgatagtgtccacttaaattttgatggacactatcacagatgacagagtccacagcccggcgcaaacacggctgtcggtcaggaaagagaaaagccagtcgctgtacgact

SEQ ID NO: 2 - protein, 322 aa, Salmonella typhimurium
MPFHIGSGCLPAIISNRRIYRIAWSDTPPEMSSWEKMKEFFCSTHQAEALECIWTICHPPAGTTREDVVSRFELLRTLAYDGWEENIHSGLHGENYFCILDEGSQEILSVTLDDVGNYTVNCQGYSETHHLTMATEPGVERTDITYNLTSDIDAAAYLEELKQNPIINNKIMNPVGQCESLMTPVSNFMNEKGFDNIRYRGIFIWDKPTEEIPTNHFAVVGNKEGKDYVFDVSAHQFENRGMSNLNGPLILSADEWVCKYRMATRRKLIYYTDFSNSSIAANAYDALPRELESESMAGKVFVTSPRWFNTFKKQKYSLIGKM

SEQ ID NO: 3 - DNA, 648 bp, Salmonella typhimurium
atggacgatgagcgcctgaaaaatccgcccgtgggttcatcggctgtacccgactattttgatgagatgctggagcgtatccgcgatattcgcgccagcgaacgtcgggtttatttgcgggtacgagagatctttgcgttagccgccgactatcaaccatcgctcaaagaaaccacgcaattttttcaaaccatccagaacaagttgcattttgcctgtaccggacataccgctgctgaactcattcatcagcgtgctgacgccagccagccgcatatggggctgaccagctataaaggtgaagaggtacgtaaggatgacgtgacggtggcaaaaaattatctcactcaggatgaagtcagcgagcttaaccgcgtagttaacatgtggctggattttgccgaggatcaggcccgtcgtcgtcagcagatctttttacgcgactggcaggataagctggatcagttcctgcaatttaacgaccgtgaggttttacaaggcgcaggtaaagtcactaagaaaatggccgatgaaaaagcgcaggcggaatatagtcagtttgctgaacaacaacggcgcttaaaagaagccgaaggtgagaaggatatcgccggtttgctacaatgggaaacagaacctaaaaagtag

SEQ ID NO: 4 - protein, 215 aa, Salmonella typhimurium
MDDERLKNPPVGSSAVPDYFDEMLERIRDIRASERRVYLRVREIFALAADYQPSLKETTQFFQTIQNKLHFACTGHTAAELIHQRADASQPHMGLTSYKGEEVRKDDVTVAKNYLTQDEVSELNRVVNMWLDFAEDQARRRQQIFLRDWQDKLDQFLQFNDREVLQGAGKVTKKMADEKAQAEYSQFAEQQRRLKEAEGEKDIAGLLQWETEPKK

SEQ ID NO: 5 - DNA, 1581 bp, Salmonella typhimurium
atgctgcagaactttttggctgacaatgtagcaaaagacaatctggctcagcaaagcgatgcttcccagcaaaatacacaggctaaagcaacgcaggcttctaaacagaacgatgctgaaaaagttcttcctcaacctattaataaaaataccagtactggcaaaagtaatagcagtaaaaatgaggaaaataagctcgatgccgagtctgttaaagagccgcttaaagtcacattagcgcgtgcggccgagagtaacagcggtagcaaagatgatagtataactaattttaccaaacctcagtttgtagttagcactgctcccaatgccacggttattattaaaattaatggtattgctgtcggtcaggctgtaacggatagtttgggtaacttcacctttacagcgcctgaaacattgactgatggaacatataatctggaggcagaggccaagactgctgatgggagcggtagcgccaaacttgtcattactatcgattccgttaccgataaaccaacatttgaactttcgcctgaaagtagtgtgtccggtcataagggcttaacgccgaccttgacgccttcaattgttggtacggcggaagagaatgctaaggttgacatttatgtagataataaactggttgccagcgttgatgtcgataaagatggaaactggagttatgaatttaaggataatgaattatctgagggcgaaaatagtataaaagtcgttgctgtagataaagcaggtaataaaaacgaaacgacggatagtatcataaccgacaccattgctccagaaaagccgacgattgagctggatgatagtagtgattccggcattaaaaatgacaacattacaaatagcaccctgccaacatttattggtgtggcggaacccggttctacagtctctatttatcttggacttaaacatcttggtgaggtcattgttgctaaagatgggacatggagctatacgcttactacgccgctcaaggatggcgaatacaatataacagcaacagctactgatattgccgggcatacctcagcgacggcaaatctgccttttactattgatacacgtatcagctatttcagcgctgagattgaaacgacgaatgatagcggtattgtcggagataacgttactaacaatactcgcccaacctttacaggtaaaactgagccaaatgctattatcagtgtcataaatagtgagactggcgaagaggttatttttaaagcgaatgacaagggcgaatggacgttcaatttcacttccgactcagtggaagggattaacaatcttacgttcactgttgaagatgtcgctggcaacaaaaaggatttttcctttagttacgttattgatactattgcccctgtacctccgacggcttctttggaggattatgttgttttgccgaatggtataattttatcagggaatgatttaccggctttagtcggtacggcagaaccaaagtctaccatcttattgatgcgagatggtaaattatatgacagcattgaagttgactcaaacgggacctggaaattatcagtttag

SEQ ID NO: 6 - protein, 526 aa, Salmonella typhimurium
MLQNFLADNVAKDNLAQQSDASQQNTQAKATQASKQNDAEKVLPQPINKNTSTGKSNSSKNEENKLDAESVKEPLKVTLARAAESNSGSKDDSITNFTKPQFVVSTAPNATVIIKINGIAVGQAVTDSLGNFTFTAPETLTDGTYNLEAEAKTADGSGSAKLVITIDSVTDKPTFELSPESSVSGHKGLTPTLTPSIVGTAEENAKVDIYVDNKLVASVDVDKDGNWSYEFKDNELSEGENSIKVVAVDKAGNKNETTDSIITDTIAPEKPTIELDDSSDSGIKNDNITNSTLPTFIGVAEPGSTVSIYLGLKHLGEVIVAKDGTWSYTLTTPLKDGEYNITATATDIAGHTSATANLPFTIDTRISYFSAEIETTNDSGIVGDNVTNNTRPTFTGKTEPNAIISVINSETGEEVIFKANDKGEWTFNFTSDSVEGINNLTFTVEDVAGNKKDFSFSYVIDTIAPVPPTASLEDYVVLPNGIILSGNDLPALVGTAEPKSTILLMRDGKLYDSIEVDSNGTWKLSV

SEQ ID NO: 7 - DNA, 579 bp, Salmonella paratyphi
  CDS (1)...(579)
atgtaccaggatcttattcgtaacgaactgaacgaagcggcggaaacgctggctaattttttaaaagatgacgccaatattcacgccattcagcgcgcggcggtcctgttggctgacagttttaaagcgggtggtaaggtactttcctgcggtaacggcggctcccattgcgatgcgatgcactttgctgaagagctgacaggtcgttatcgtgaaaatcgtccgggctacccggcaattgcgatttctgatgttagccatatatcctgcgttagtaatgattttggctatgactatattttctctcgttacgttgaggcggtagggcgcgaaggcgatgtcctgttagggatctcgacgtcgggtaactctggtaacgtgattaaagcgattgccgcggcccgtgaaaaaggcatgaaagtgatcacgttgaccggtaaagacggcggcaaaatggcgggaacggcggatattgaaattcgcgtaccgcacttcggttatgccgatcgtattcaggaaattcatatcaaagtaattcatatcctgattcagttgatcgaaaaagagatggttaaataa

SEQ ID NO: 8 - protein, 192 aa, Salmonella paratyphi
MYQDLIRNELNEAAETLANFLKDDANIHAIQRAAVLLADSFKAGGKVLSCGNGGSHCDAMHFAEELTGRYRENRPGYPAIAISDVSHISCVSNDFGYDYIFSRYVEAVGREGDVLLGISTSGNSGNVIKAIAAAREKGMKVITLTGKDGGKMAGTADIEIRVPHFGYADRIQEIHIKVIHILIQLIEKEMVK

SEQ ID NO: 9 - DNA, 299 bp, Salmonella typhimurium
atgcggccaataaaaaatgctaaaaaaattgactacaatctgatcaaagtgttcgatacggttattactgaaggaaatgcaaccagggcggcgaggaaactggatgtcacgcctgcggcgatctctcaggctcttcttcgtttacaaaatctttatggcgaagagttatttatcagaacccgcaaaggattagttccgtccagcaaaggtaaatcgcttcaccaggtatttcgccaggcaattgaatctatagaaagcacactgtgcgataaaacagatgctcaggagagtaatgaacc

SEQ ID NO: 10 - protein, 100 aa, Salmonella typhimurium
MRPIKNAKKIDYNLIKVFDTVITEGNATRAARKLDVTPAAISQALLRLQNLYGEELFIRTRKGLVPSSKGKSLHQVFRQAIESIESTLCDKTDAQESNEP

SEQ ID NO: 11 - DNA, 1224 bp, Salmonella typhimurium
ggagaaaagatgctaaccacatcattaacgttaaataaagagaaatggaagccgatctggaataaagcgctggtttttctttttgttgccacgtattttctggatggtattacgcgttataaacatttgataatcatacttatggttatcaccgcgatttatcaggtctcacgctcaccgaaaagtttcccccctcttttcaaaaatagcgtattttatagcgtagcagtattatcattaatccttgtttattccatactcatatcgccagatatgaaagaaagtttcaaggaatttgaaaatacggtactggagggcttcttattatatactttattaattcccgtactattaaaagatgaaacaaaagaaacggttgcgaaaatagtacttttctcctttttaacaagtttaggacttcgctgccttgcagagagtattctgtatatcgaggactataataaagggattatgccattcataagctatgcgcatcgacatatgtccgattccatggttttcttatttccagcattattgaatatttggctgtttagaaaaaatgcaattaagttggtttttttggtgcttagcgccatctaccttttctttatcctgggaaccctatcgcgaggggcatggttggcggtgcttatagtaggtgttctgtgggcaatactgaaccgccaatggaagttaataggagttggtgccattttattagccattatcggcgctttggttatcactcaacataataacaaaccagacccagaacatttactgtataaattacagcagacagatagctcatatcgttatactaacggaacccagggcaccgcgtggatactgattcaggaaaacccgatcaagggctacggctatggtaatgatgtgtatgatggtgtttataataaacgcgttgtcgattatccaacgtggacctttaaagaatctatcggtccgcataataccattctgtacatctggtttagtgcaggcatattgggtctggcgagcctggtctatttatatggcgctatcatcagggaaacagccagctctaccctcaggaaagtagagataagcccctacaatgctcatctcttgctatttttatctttcgtcggtttttatatcgttcgtggcaattttgaacaggtcgatattgctcaaattggtatcattaccggttttctgctggcgctaagaaatagataa

SEQ ID NO: 12 - protein, 404 aa, Salmonella typhimurium
MLTTSLTLNKEKWKPIWNKALVFLFVATYFLDGITRYKHLIIILMVITAIYQVSRSPKSFPPLFKNSVFYSVAVLSLILVYSILISPDMKESFKEFENTVLEGFLLYTLLIPVLLKDETKETVAKIVLFSFLTSLGLRCLAESILYIEDYNKGIMPFISYAHRHMSDSMVFLFPALLNIWLFRKNAIKLVFLVLSAIYLFFILGTLSRGAWLAVLIVGVLWAILNRQWKLIGVGAILLAIIGALVITQHNNKPDPEHLLYKLQQTDSSYRYTNGTQGTAWILIQENPIKGYGYGNDVYDGVYNKRVVDYPTWTFKESIGPHNTILYIWFSAGILGLASLVYLYGAIIRETASSTLRKVEISPYNAHLLLFLSFVGFYIVRGNFEQVDIAQIGIITGFLLALRNR

SEQ ID NO: 13 - DNA, 2119 bp, Salmonella typhimurium
acggacaacaactatgaataaatcagggaaatacctcgtctggacagcgctctcagtattgggtgcgtttgccctgggctatattgcgttaaatcgtggggaacagatcaacgcgctatggatcgtggtggcgtcggtctgtgtctatcttattgcgtatcgtttttatgggctctatatcgccaaaaaagtgctggcggttgacccaacgcgtatgacgcccgcggtacgtcataatgatggtctggattatgtcccgaccgataaaaaagtgctgttcggtcaccattttgcggccattgctggcgcaggtccgctggtcgggccggtactggcggcgcagatgggctatctgccggggatgatctggctgctggcgggcgtcgtgctggcgggagcggtgcaggactttatggtgctgttcgtctcgacccggcgcgatgggcgttcgcttggcgagctggttaaagaggagatgggcgcgacggcaggggtgatcgcgctggtggcctgctttatgatcatggtgatcattctggccgtcctggcgatgatcgtggtgaaagcgctgacccatagcccgtggggaacgtacactgtcgcgttcactattccactggcgatttttatgggcatctacttgcgttatttgcgtccggggcgcatcggcgaggtgtcggtcattgggctggtatttcttattttcgctattatttccggcggatgggtggcggcaagcccaacctgggcgccgtactttgattttactggcgtgcagcttacctggatgctggtgggttatggttttgtcgcggcggtactgccggtctggctgctgctcgcgccgcgtgattacctctctaccttcctgaaaattggtacgattgtcggtctggcggtcgggattctgattatgcgtccgacgctgactatgccggcgctgaccaaatttgttgatggtaccggaccggtctggacgggcgacctgttcccgttcctgtttattaccatcgcctgcggcgcggtctccggtttccatgcgctcatctcctccggcacgacgccgaagatgttggccaacgaaggccaggcctgctttatcggctacggcgggatgttaatggaatctttcgtcgccattatggcgctggtctccgcctgtattatcgatccgggtgtttactttgcgatgaatagcccgatggcggtactggcgccagcggggacagcggatgtcgtagcttctgccgcgcaggtggtcagtagttggggtttcgctatcacgccggatacgttacaccagattgccaatgaagtcggcgaacaatccattatctcccgcgcaggcggagcgccaacgctggcggtagggatggcctacattttacatggcgcgttgggcggcatgatggatgtggcgttctggtatcacttcgccattctgtttgaagcgctgtttattctgacggcggtggatgcgggcacccgtgcggcgcgctttatgttgcaggatttgttgggcgtagtgtcgccagggctgaaacgtaccgattcgttgccagcgaacctgcttgctacggcattgtgcgtgctggcgtgggggtatttcctccatcagggcgtggtcgatccgttgggcggtattaacaccctgtggccgctgtttggcatcgctaaccagatgctggcgggtatggcgctgatgctttgcgccgtggtactgttcaaaatgaagcgtcagcgttatgcgtgggtcgcgctggtgccgacggcctggctgctgatttgtacgctgacggcgggttggcagaaagcgtttagtccggatgcgaaaatcggcttcctggccattgccaataagttccaggcgatgatcgacagcggcaatattccgccgcaatacaccgaatcgcaactcgcgcagttggtattcaataaccgtctggatgccgggctaaccatcttctttatggtggtggtcgtggtgctggcggtcttctctattaagacggcgctggccgctctgaagattgataaaccgacggcgaatgaaacgccgtatgagccgatgccggaaaatgtggatgagatcgtgacgcaggcgaaaggcgcgcactaa

SEQ ID NO: 14 - protein, 701 aa, Salmonella typhimurium
MNKSGKYLVWTALSVLGAFALGYIALNRGEQINALWIVVASVCVYLIAYRFYGLYIAKKVLAVDPTRMTPAVRHNDGLDYVPTDKKVLFGHHFAAIAGAGPLVGPVLAAQMGYLPGMIWLLAGVVLAGAVQDFMVLFVSTRRDGRSLGELVKEEMGATAGVIALVACFMIMVIILAVLAMIVVKALTHSPWGTYTVAFTIPLAIFMGIYLRYLRPGRIGEVSVIGLVFLIFAIISGGWVAASPTWAPYFDFTGVQLTWMLVGYGFVAAVLPVWLLLAPRDYLSTFLKIGTIVGLAVGILIMRPTLTMPALTKFVDGTGPVWTGDLFPFLFITIACGAVSGFHALISSGTTPKMLANEGQACFIGYGGMLMESFVAIMALVSACIIDPGVYFAMNSPMAVLAPAGTADVVASAAQVVSSWGFAITPDTLHQIANEVGEQSIISRAGGAPTLAVGMAYILHGALGGMMDVAFWYHFAILFEALFILTAVDAGTRAARFMLQDLLGVVSPGLKRTDSLPANLLATALCVLAWGYFLHQGVVDPLGGINTLWPLFGIANQMLAGMALMLCAVVLFKMKRQRYAWVALVPTAWLLICTLTAGWQKAFSPDAKIGFLAIANKFQAMIDSGNIPPQYTESQLAQLVFNNRLDAGLTIFFMVVVVVLAVFSIKTALAALKIDKPTANETPYEPMPENVDEIVTQAKGAH

SEQ ID NO: 15 - DNA, 19 bp, Salmonella typhimurium
gcagaaacgttgggattgc

SEQ ID NO: 16 - DNA, 22 bp, Salmonella typhimurium
actggaggattgtaccattgca

SEQ ID NO: 17 - DNA, 681 bp, Salmonella enterica serovar Dublin
  CDS (1)...(681)
atgcttccggtcacctacagattaatacctcaaagcggagtatccacatatggattaaataccgcagatacacctgttttccccgatattcccgaacatgcaccaaacccctccatgctacgccttgctcatgacagccttgccataaacagtgaattccgtctggagccagagtgtgtggtggagtaccttatctcaggcgcgggtggaatagaccctgatacagaaattgatgacgacacttataacgaatgctacgatgaactatcctccgtacttcaaaatgcgtatacccaaagcgaaacattccgcagactgatgaattacgcatatgaaaaagaactacatgatgtggagcagcgctggctactgggggcaggcgaagcctttgaaacttccgtggctcaggaacacttcaaactttcagaaggcaggaaagttatttgtctcaatctggacgattctgatgattcatataccgaacattatgaaagtaacgaaggaccacaactttttgacacaaaacgttcatttattcatgaagttgtacatgcactgacccatcttcaggataaagaagaaaatcatccaagaggccctgttgtcgaatataccaacattattctgaaagagatggggcatccttcacctcccagaatggcctacatcttcaataaatag

SEQ ID NO: 18 - protein, 226 aa, Salmonella enterica serovar Dublin
MLPVTYRLIPQSGVSTYGLNTADTPVFPDIPEHAPNPSMLRLAHDSLAINSEFRLEPECVVEYLISGAGGIDPDTEIDDDTYNECYDELSSVLQNAYTQSETFRRLMNYAYEKELHDVEQRWLLGAGEAFETSVAQEHFKLSEGRKVICLNLDDSDDSYTEHYESNEGPQLFDTKRSFIHEVVHALTHLQDKEENHPRGPVVEYTNIILKEMGHPSPPRMAYIFNK

SEQ ID NO: 19 - DNA, 20 bp, Salmonella typhimurium
tcgtatcggttgataccggc

SEQ ID NO: 20 - DNA, 20 bp, Salmonella typhimurium
gaacctatgtcgagcgacag

SEQ ID NO: 21 - DNA, 20 bp, Salmonella typhimurium
ctgaccacttgtgatgatta

SEQ ID NO: 22 - DNA, 20 bp, Salmonella typhimurium
agcagatatccgtcggcaca

(19) United States
(12) Patent Application Publication
(10) Pub. No.: US 2004/0253712 A1
(43) Pub. Date: Dec. 16, 2004

(54) Salmonella virulence factors and uses thereof

(76) Inventors: Frederick M. Ausubel, Newton, MA (US); Alejandro Aballay, Durham, NC (US); Eliana Drenkard, Cambridge, MA (US); Beth A McCormick, Hevrhill, MA (US)

Correspondence Address: CLARK & ELBING LLP, 101 FEDERAL STREET, BOSTON, MA, 02110, US

(21) Appl. No.: 10/494,087

(22) PCT Filed: Nov. 1, 2002
(86) PCT No.: PCT/US02/35039

Related U.S. Application Data
(60) Provisional application No. 60/334,761, filed on Nov. 1, 2001.

(51) Int. Cl. C12N 1/20

(52) U.S. Cl. 435/252.3

(57) ABSTRACT

The invention features nucleic acid molecules encoding microbial virulence factors (such as gmhA) and methods of using such gene (and their proteins) as targets to identify anti-pathogenic agents. The invention also features a method of identifying a compound that inhibits the pathogenicity of an effector protein in a nematode, the method involving the steps of: (a) providing a nematode expressing an effector protein; (b) contacting the nematode with a test compound; and (c) determining whether the test compound inhibits the pathogenicity of the effector polypeptide in the nematode.

### CROSS-REFERENCE TO RELATED APPLICATIONS

This application is the U.S. National Stage of International Application No. PCT/US02/35039, filed Nov. 1, 2002, which was published in English under PCT Article 21(2), and which claims priority from U.S. Ser. No. 60/334,761 filed Nov. 1, 2001.

### BACKGROUND OF THE INVENTION

The invention relates to microbial virulence factors and uses thereof.

The use of genetic techniques to identify bacterial virulence factors involved in mammalian pathogenesis is often complicated by the tediousness, expense, and ethical considerations of using large numbers of vertebrate animals to identify avirulent bacterial mutants. To overcome this problem, two genetic methods, in vivo expression technology and signature tagged mutagenesis (reviewed in Strauss et al., Science 276:707-712, 1997) have been used to identify bacterial virulence factors (Lehoux et al., Biotechniques 26:473-478,1999; Wang et al., Mol. Microbiol. 22:1005-1012, 1996; Wang et al., Proc. Natl. Acad. Sci. 93:10434-10439, 1996). These methods allow the application of random mutagenesis and high throughput screening for the identification of bacterial virulence factors in the context of actual host environments. This represents a complementary approach to in vivo expression technology and signature tagged mutagenesis that involves the use of non-vertebrate hosts including the plant Arabidopsis thaliana or the nematode Caenorhaditis elegans that can be infected and killed by common human bacterial pathogens such as Pseudomonas aeruginosa (Tan et al., Proc. Natl. Acad. Sci. 96:715-720, 1999; Tan et al., Proc. Natl. Acad. Sci. 96:2408-2113, 1999; Mahajan-Miklos et al., Cell 96:47-56, 1999), Enterococcus faecalis (Garsin et. al., Proc. Natl. Acad. Sci. 98:10892-10897, 2001), or Salmonella enterica (Aballay et al., Curr. Biol. 98:2735-2739, 2001; Aballay et al., Curr. Biol. 10:1539-1542, 2000; Labrousse et al., Curr. Biol. 10:1543-1545, 2000). For example, screening a P. aeruginosa library of transposon insertion mutants for loss of infectivity in plants or nematodes led to the identification of 22 putative P. aeruginosa virulence factors, 18 of which were also required for full pathogenesis in a mouse burn model (Rahme et al., Proc. Natl. Acad. Sci. 94:13245-13250, 1997; Tan et al., Proc. Natl. Acad. Sci. 96:715-720, 1999; Tan et al., Proc. Natl. Acad. Sci. 96:2408-2413, 1999; Mahajan-Miklos et al., 96:47-56, 1999).

The C elegans-Salmonella model is of particular interest because Salmonella serovars such as S. typhimurium kill not only C. elegans, but in contrast to P. aeruginosa, are also capable of establishing a persistent infection in the C elegans intestine (Aballay et al., Curr. Biol. 10:1539-1542, 2000; Labrousse et al., Curr. Biol. 10:1543-1545, 2000). Moreover, an intact PhoP/PhoQ signal transduction system, a major regulator of virulence-related genes in vertebrate hosts, as well as the fur-1 and ompR genes, which are known to be involved in different aspects of acid tolerance in S. enterica, are required for full pathogenicity in C. elegans (Aballay et al., Curr. Biol. 10:1539-1542, 2000; Labrousse et al., Cur. Biol. 10:1543-1545, 2000).

### SUMMARY OF THE INVENTION

As is described below, we have identified and characterized a number of nucleic acid molecules and polypeptides that are involved in conferring pathogenicity and virulence to a pathogen. This discovery therefore provides a basis for drug-screening assays aimed at evaluating and identifying “anti-virulence” agents which are capable of blocking pathogenicity and virulence of a pathogen, e.g., by selectively switching pathogen gene expression on or off, or which inactivate or inhibit the activity of a polypeptide which is involved in the pathogenicity of a microbe. Drugs that target these molecules are useful as such anti-virulence agents.

Accordingly, the invention features an isolated nucleic acid molecule including a sequence substantially identical to any one of the nucleic acid sequences encoding srfH (SEQ ID NO:1), rhuM (SEQ ID NO: 3), spi4-F (SEQ ID NO:5), gmhA (SEQ ID NO:7), leuO (SEQ ID NO:9), rfaL (SEQ ID NO:11), cstA (SEQ ID NO:13), and pipA (SEQ ID NO:17). Preferably, the isolated nucleic acid molecule includes any of the above-described sequences or a fragment thereof; and is derived from a pathogen (e.g., from a bacterial pathogen such as Salmonella). Additionally, the invention includes a vector and a cell, each of which includes at least one of the isolated nucleic acid molecules of the invention; and a method of producing a recombinant polypeptide involving providing a cell transformed with a nucleic acid molecule of the invention positioned for expression in the cell, culturing the transformed cell under conditions for expressing the nucleic acid molecule, and isolating a recombinant polypeptide. The invention further features recombinant polypeptides produced by such expression of an isolated nucleic acid molecule of the invention, and substantially pure antibodies that specifically recognize and bind such a recombinant polypeptide.

In an another aspect, the invention features a substantially pure polypeptide including an amino acid sequence that is substantially identical to the amino acid sequence of SrfH (SEQ ID NO:2), RhuM (SEQ ID NO:4), Spi4-F (SEQ ID NO:6), GmhA (SEQ ID NO:8), LeuO (SEQ ID NO:10), RfaL (SEQ ID NO:12), CstA (SEQ ID NO:14), and PipA (SEQ ID NO: 18). Preferably, the substantially pure polypeptide includes any of the above-described sequences or a fragment thereof; and is derived from a pathogen (e.g., from a bacterial pathogen such as Salmonella).

In yet another related aspect, the invention features a method for identifying a compound which is capable of decreasing the expression of a pathogenic virulence factor (e.g., at the transcriptional or post-transcriptional levels), involving the steps of (a) providing a pathogenic cell (e.g., a microbial cell) expressing any one of the isolated nucleic acid molecules of the invention; and (b) contacting the pathogenic cell with a candidate compound, a decrease in expression of the nucleic acid molecule following contact with the candidate compound identifying a compound which decreases the expression of a pathogenic virulence factor. In preferred embodiments, the pathogenic cell infects a mammal (e.g., a human).

In yet another related aspect, the invention features a method for identifying a compound which binds a polypeptide, involving (a) contacting a candidate compound with a substantially pure polypeptide including any one of the amino acid sequences of the invention (e.g., the amino acid sequence of SrfH (SEQ ID NO:2), RhuM (SEQ ID NO:4), Spi4-F (SEQ ID NO:6), GmhA (SEQ ID NO:8), LeuO (SEQ ID NO:10), RfaL (SEQ ID NO:12), CstA (SEQ ID NO:14), and PipA (SEQ ID NO: 18)) under conditions that allow binding; and (b) detecting binding of the candidate compound to the polypeptide.

In yet another aspect, the invention features a method of treating a pathogenic infection in a mammal, involving (a) identifying a mammal having a pathogenic infection; and (b) administering to the mammal a therapeutically effective amount of a composition which binds a polypeptide encoded by any one of the amino acid sequences of the invention (e.g., the amino acid sequence of SrfH (SEQ ID NO:2), RhuM (SEQ ID NO:4), Spi4-F (SEQ ID NO:6), GmhA (SEQ ID NO:8), LeuO (SEQ ID NO:10), RfaL (SEQ ID NO:12), CstA (SEQ ID NO:14), and PipA (SEQ ID NO: 18)). In preferred embodiments, the pathogenic infection is caused bacteria such as Aerobacter, Aeromonas, Acinetobacter, Agrobacterium, Bacillus, Bacteroides, Bartonella, Bordetella, Bortella, Borrelia, Brucella, Burkholderia, Calymmatobacterium, Campylobacter, Citrobacter, Clostridium, Cornyebacterium, Enterobacter, Enterococcus, Escherichia, Francisella, Gardnerella, Haemophilus, Hafnia, Helicobacter, Klebsiella, Legionella, Listeria, Morganella, Moraxella, Mycobacterium, Neisseria, Pasteurella, Proteus, Providencia, Pseudomonas, Salmonella, Serratia, Shigella, Staphylococcus, Streptococcus, Stentorophomonas, Treponema, Xanthomonas, Vibrio, and Yersinia.

By “virulence factor” is meant a cellular component (e.g., a protein such as a transcription factor or a molecule) without which a pathogen is incapable of causing disease or infection in a eukaryotic host organism (e.g., a nematode or mammal). Such components are involved in the adaptation of the bacteria to a host (e.g., a nematode host), establishment of a bacterial infection, maintenance of a bacterial infection, and generation of the damaging effects of the infection to the host organism. Further, the phrase includes components that act directly on host tissue, as well as components which regulate the activity or production of other pathogenesis factors.

By “infection” or “infected” is meant an invasion or colonization of a host animal (e.g., nematode) by pathogenic bacteria that is damaging to the host.

By “inhibits pathogenicity of a Salmonella pathogen” is meant the ability of a test compound to decrease, suppress, attenuate, diminish, arrest, or stabilize the development or progression of a Salmonella-induced disease or infection in a eukaryotic host organism. Preferably, such inhibition decreases pathogenicity by at least 5%, more preferably by at least 25%, and most preferably by at least 50% or more, as compared to symptoms in the absence of the test compound in any appropriate pathogenicity assay (for example, those assays described herein). In one particular example, inhibition may be measured by monitoring pathogenic symptoms in a nematode infected with a Salmonella pathogen exposed to a test compound or extract, a decrease in the level of pathogenic symptoms relative to the level of symptoms in the host organism not exposed to the compound indicating compound-induced inhibition of the Salmonella pathogen.

By “a component of a MAPK signaling pathway” is meant a polypeptide with identity to the mitogen-activated protein kinases (MAPK) or an polypeptide with identity to a MAPKK or MAPKKK. The core of a MAPK signaling pathway is composed of a MAP kinase (MAPK) (such as p38, JNKs, Jun amino-terminal kinases, or ERKs, extracellular signal-related kinases) whose activity is regulated via a MAPK-activating MAPK kinase (MAPKK) (such as MKK3/6, MKK4/7, or MEK1/2), which, in turn, is activated by a MAPKK-activating MAPKK kinase (MAPKKK) (such as ASK1 or c-Raf).

By “a mutated MAPK signaling pathway” is meant a MAPK signaling pathway having an alteration that enhances or diminishes a nematode innate immune response. Such an alteration might include without limitation the genetic inhibition of a MAPK (or a MAPKK or MAPKKK) by chemical or transposon-mediated mutagenesis, interference with MAPK gene expression by RNA-mediated interference, or the expression of a MAPK transgene, such a transgene might overexpress or interfere with a MAPK signaling component.

By “innate immunity” is meant a native or natural immunity whose defense mechanisms are present prior to exposure to infectious microbes or foreign substances.

By “isolated nucleic acid molecule” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

By “polypeptide” is meant any chain of amino acids, regardless of length or post-translational modification (for example, glycosylation or phosphorylation).

By a “substantially pure polypeptide” is meant a polypeptide of the invention that has been separated from components which naturally accompany it. Typically, the polypeptide is substantially pure when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. A substantially pure polypeptide of the invention may be obtained, for example, by extraction from a natural source (for example, a pathogen); by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “substantially identical” is meant a polypeptide or nucleic acid molecule exhibiting at least 25% identity to a reference amino acid sequence (for example, any one of the amino acid sequences described herein) or nucleic acid sequence (for example, any one of the nucleic acid sequences described herein). Preferably, such a sequence is at least 30%, 35%, 40%, 45%, 50%, 55%, 60%, or 65% identical, more preferably 70%, 75%, or over 80% identical, and most preferably 90% or even 95% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “transformed cell” is meant a cell into which (or into an ancestor of which) has been introduced, by means of recombinant DNA techniques, a DNA molecule encoding (as used herein) a polypeptide of the invention.

By “positioned for expression” is meant that the DNA molecule is positioned adjacent to a DNA sequence which directs transcription and translation of the sequence (i.e., facilitates the production of, for example, a recombinant polypeptide of the invention, or an RNA molecule).

By “purified antibody” is meant antibody which is at least 60%, by weight, free from proteins and naturally-occurring organic molecules with which it is naturally associated. Preferably, the preparation is at least 75%, more preferably 90%, and most preferably at least 99%, by weight, antibody. A purified antibody of the invention may be obtained, for example, by affinity chromatography using a recombinantly-produced polypeptide of the invention and standard techniques.

By “specifically binds” is meant a compound or antibody which recognizes and binds a polypeptide of the invention but which does not substantially recognize and bind other molecules in a sample, for example, a biological sample, which naturally includes a polypeptide of the invention.

By “derived from” is meant isolated from or having the sequence of a naturally-occurring sequence (e.g., a cDNA, genomic DNA, synthetic, or combination thereof).

The present invention facilitates the identification of novel targets and therapeutic approaches for preparing therapeutic agents active on Salmonella virulence factors and genes. The invention also provides long awaited advantages over a wide variety of standard screening methods used for distinguishing and evaluating the efficacy of a compound against Salmonella pathogens. In one particular example, the screening methods described herein allow for the simultaneous evaluation of host toxicity as well as anti-Salmonella potency in a simple in vivo screen. Moreover, the methods of the invention allow one to evaluate the ability of a compound to inhibit Salmonella pathogenesis, and, at the same time, to evaluate the ability of the compound to stimulate and strengthen a host's response to Salmonella pathogenic attack.

In addition, the invention provides methods for the identification of host genes required for pathogen defense, such genes function in innate immunity.

Accordingly, the methods of the invention provide a straightforward means to identify compounds that are both safe for use in eukaryotic host organisms (i.e., compounds which do not adversely affect the normal development and physiology of the organism) and efficacious against Salmonella pathogenic microbes. In addition, the methods of the invention provide a route for analyzing virtually any number of compounds for anti-Salmonella pathogenic effect with high-volume throughput, high sensitivity, and low complexity. The methods are also relatively inexpensive to perform and enable the analysis of small quantities of active substances found in either purified or crude extract form. Furthermore, the methods disclosed herein provide a means for identifying anti-pathogenic compounds which have the capability of crossing eukaryotic cell membranes and which maintain therapeutic efficacy in an in vivo method of administration. In addition, the above-described methods of screening are suitable for both known and unknown compounds and compound libraries.

Other features and advantages of the invention will be apparent from the following description of the preferred embodiments thereof, and from the claims.

### BRIEF DESCRIPTION OF THE DRAWINGS

FIGS. 1A-1C show the pathogenicity phenotypes of S. enterica strains containing mutations in SPI-1 genes. FIG. 1A shows C. elegans glp-4 young adult animals susceptibility to S. enterica SLR2 or E. coli OP50 (P<0.0002), and C. elegans N2 (wild-type) young adult animals susceptibility to S. enterica SLR2 or E. coli OP50 (P<0.003). FIG. 1B shows C. elegans glp-4 young adult animals susceptibility to S. enterica SL1344, LM399 (hilD) (P<0.036), LS666 (hilD/hilC) (P<0.032), or SD11 (Δspi-1) (P<0.016). FIG. 1C shows C. elegans N2 (wild-type) young animals susceptibility to S. enterica SL1344, S. enterica SL1344 (pVV214) overexpressing HilA (P<0.048), or S. enterica SL1344 invF::Tn5lacZY(pVV214) (P<0.82). Twenty animals were used in each case. Nematode survival was plotted using the PRISM (version 2.00) computer program. Survival curves were considered significantly different from a glp-4 exposed to S. enterica SLR2 control, a glp-4 exposed to S. enterica SL1344 control, or from a N2 exposed to S. enterica SL1344 control when P values were <0.05.

FIGS. 2A-2F show the pathogenicity phenotypes of S. enterica TnphoA mutants. FIG. 2A shows C. elegans glp-4 young adult animals susceptibility to S. enterica SLR2 or to different SLR2 TnphoA insertion mutants: 9D5 (invH) (P<0.002), 7D2 (hilD) (P<0.014), or 1E1 (hilA) (P<0.007). FIG. 2B shows C. elegans glp-4 young adult animals susceptibility to S. enterica SLR2 or to different SLR2 TnphoA insertion mutants: 3E4 (srfH) (P<0.0002), 2C4 (sptP) (P<0.048). FIG. 2C shows C. elegans glp-4 young adult animals susceptibility to S. enterica SLR2 or to different SLR2 TnphoA insertion mutants:3E11 (rhuM) (P<0.004), 8F4 (pipA) (P<0.022), or 6E5 (spi4-F) (P<0.014). FIG. 2D shows C. elegans glp-4 young adult animals susceptibility to S. enterica SLR2 or to different SLR2 TnphoA insertion mutants: 2A6 (vsdA) (P<0.013), 4C4 (copB/repC) (P<0.042). FIG. 2E shows C. elegans glp-4 young adult animals susceptibility to S. enterica SLR2 or to different SLR2 TnphoA insertion mutants:6D4 (gmhA) (P<0.011) or 3H7 (rfaL) (P<0.0002). FIG. 2F shows C. elegans glp-4 young adult animals susceptibility to S. enterica SLR2 or to different SLR2 TnphoA insertion mutants: 5F3 (leuO) (P<0.003), 7A2 (recB) (P<0.008), or 2A4 (cstA) (P<0.043). Twenty animals were used in each case. Nematode survival was plotted using the PRISM (version 2.00) computer program. Survival curves were considered significantly different from the glp-4+S. enterica SLR2 control when P values were <0.05.

FIGS. 3A-3B are photographs showing GFP expression in C. elegans. FIG. 3A is a photograph showing GFP expression in an adult transgenic nematode carrying a kin-18::GFP reporter construct. FIG. 3B is a photograph showing GFP expression in an arrested nematode embryo carrying a kin-18::srfH::GFP reporter construct. GFP expression is indicated with an arrow.

FIGS. 4A-4C show that intact Salmonella lipopolysacharide is required for persistent infection and induction of SEK-1-dependent host innate immune responses in C. elegans. FIG. 4A shows wild-type and pmk-1 mutant C. elegans susceptibility to Salmonella having wild-type or mutant lipooligosacharide. N2 young adult animals were exposed to S. enterica SL1344, E. coli OP50 (P<0.0001 compared to SL1344), S. enterica 6D4 (gmhA) (P<0.005), and S. enterica 3H7 (rfaL) (P<0.0001). Also, pmk-1 RNAi young adult animals were exposed to E. coli OP50 (P<0.0001), S. enterica 6D4 (gmhA) (P<0.0007), and S. enterica 3H7 (rfaL) (P<0.004). Twenty animals were used in each case. Nematode survival was plotted using the PRISM (version 2.00) computer program. Survival curves were considered significantly different from S. enterica SL1344 when P values were <0.05. FIG. 4B shows Salmonella titer in the intestine of C. elegans. One hundred to one hundred twenty one-day-old adult hermaphrodite worms were seeded on bacterial lawns and allowed to feed on S. enterica SL1344 (control), S. enterica 6D4 (gmhA), S. enterica 3H7 (rfaL), S. enterica 6D4 expressing gmhA (6D4/pgmhA), and S. enterica 3H7 expressing rfaL (3H7/prfaL). After twenty hours, the worms were transferred to plates containing E. coli OP50 for fifteen minutes and then transferred to new plates containing OP50. The worms were transferred to new plates every twenty-four hours thereafter, and the number of S. enterica cells associated with individual worms was determined. FIG. 4C shows the percentage of cell corpses present in N2, nsy-1, sek-1, or pmk-1 RNAi young adult hermaphrodites exposed to S. enterica SL1344, S. enterica 6D4 (gmhA), or S. enterica 3H7 (rfaL). Cell corpses were counted twenty-four hours after Salmonella exposure. Data (mean±SD) were obtained from two independent experiments and more than fifteen C. elegans were scored in each case.

FIGS. 5A-5C show the effects of Salmonella on the cell death pathway in C. elegans. FIG. 5A shows the percentage of cell corpses observed in N2, nsy-1, sek-1, or pmk-1 RNAi young adult animals exposed to E. coli OP50 or S. enterica SL1344. Cell corpses were counted twenty-four hours after the initial exposure as previously described (Gumienny et al., Development 126:1011-1022, 1999). Data (mean±SD) were obtained from two independent experiments and more than fifteen animals were scored in each case. FIG. 5B shows susceptibility of C. elegans N2 or pmk-1 RNAi young adult animals exposed to S. enterica SL1344 (P<0.039). Twenty animals were used in each case. Nematode survival was plotted using the PRISM (version 2.00) computer program. A P value <0.05 was considered significant. FIG. 5C shows the percentage of cell corpses observed in N2, ced-9, or ced-9 RNAi pmk-1 young adult hermaphrodites exposed to S. enterica SL1344. Cell corpses were counted twenty-four hours after exposure. Data (mean±SD) were obtained from two independent experiments and more than fifteen animals were scored in each case.

FIG. 6 shows the virulence phenotypes of Salmonella enterica TnphoA mutants in a typhoid mouse model. Four to six week-old female BALB/c mice (ten mice per mutant) were inoculated orally with SLR2 or with different SLR2 TnphoA insertion mutants (10⁷ CFU/mouse). Disease progression was monitored for seventeen days.

### DETAILED DESCRIPTION

As described in more detail below, we have used C. elegans-based screens to identify (i) virulence factors and microbial effectors important in mammalian pathogenesis and (ii) host pathogen defense genes.

A C. elegans-based screen was carried out to identify novel Salmonella virulence factors. S. enterica genes important in mammalian pathogenesis such as Salmonella pathogenicity island 1 (SPI-1) genes were shown to be involved in S. enterica-induced killing of C. elegans. A screen of 960 TnphoA insertion mutants yielded fifteen mutants that exhibited attenuated killing of C. elegans. Four TnphoA insertions were in the SPI-1 Type III secretion virulence-related genes invH, hilD, hilA, and sptP. One TnphoA insertion was in the srfH (SEQ ID NO:1) gene that encodes a putative effector protein, three insertions were in the rhuM (SEQ ID NO: 3), spi4-F (SEQ ID NO:5), and pipA genes located in pathogenicity islands SPI-3, SPI-4, and SPI-5, respectively, and two insertions were in the spvA and copB/copC genes located on the Salmonella virulence plasmid. Mutants carrying insertions in cstA (SEQ ID NO:13), srfH, rfaL (SEQ ID NO:11), leuO (SEQ ID NO:9), gmhA (SEQ ID NO:7), spi4-F, and pipA exhibited reduced polymorphonuclear leukocyte migration in a tissue culture model.

In addition, we show the involvement of S. enterica type III secretion-related genes in C. elegans killing and that the SrfH effector protein is an essential S. enterica virulence determinant which arrests development when expressed in C. elegans.

The role of programmed cell death in C. elegans innate immunity was also established by identifying both C. elegans and S. enterica factors that affect Salmonella-induced programmed cell death. Salmonella-induced programmed cell death was shown to require the C. elegans homologue of the mammalian p38 mitogen-activated protein kinase (MAPK) encoded by the pmk-1 gene. Inactivation of pmk-1 by RNAi blocked Salmonella-induced programmed cell death and epistasis analysis showed that CED-9 lies downstream of pmk-1. Wild-type Salmonella lipopolysaccharide (LPS) was also shown to be required for the induction of programmed cell death as well as for persistence of Salmonella in the C. elegans intestine. However, a presumptive C. elegans TOLL signaling pathway did not appear to be required for the programmed cell death response to Salmonella. These results established a PMK-1-dependent programmed cell death pathway as a C. elegans innate immune response to Salmonella.

In addition, Salmonella mutants in rfaL and gmhA were found to be avirulent in a typhoid mouse model.

The following examples are intended to illustrate, not limit, the scope of the invention.

The C. elegans Sterile Mutant glp-4 Enhances the Throughput of the Salmonella-C. elegans Killing Assay

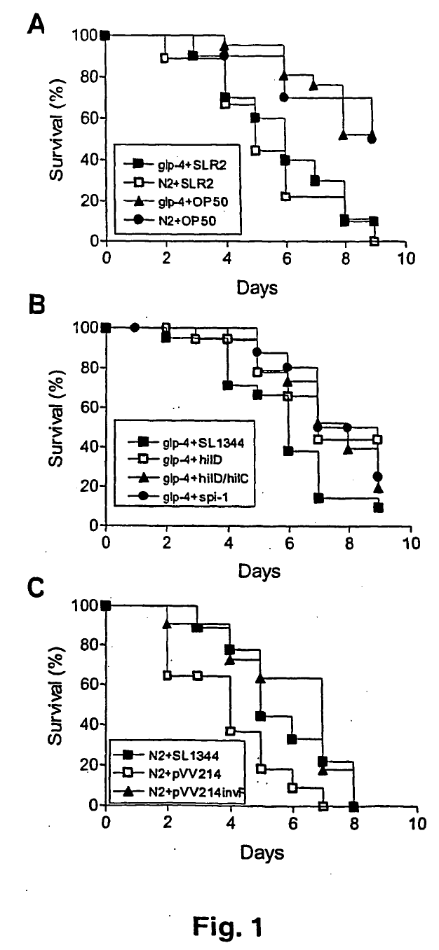

A technical difficulty associated with the C. elegans-S. enterica model arises as a consequence of the fact that the rate of C. elegans killing is slower than the generation time of C. elegans. Thus, during the first 4-5 days of the assay, the nematodes initially exposed to S. enterica had to be transferred each day to fresh plates to avoid losing track of them among their progeny. This was tedious and time-consuming, greatly reducing the number of assays that could be performed. We therefore tested whether temperature sensitive C. elegans glp-4 worms, in which germ cells are blocked in mitotic prophase and fail to proliferate and differentiate at the restrictive temperature of 25° C., could be substituted for wild-type N2 animals in the S. enterica killing assay. As shown in FIG. 1A, glp-4 worms exhibit a similar susceptibility to Salmonella-induced killing when compared to wild-type worms, although 10-20% of glp-4 worms remained alive 9 days after S. enterica infection. This result is consistent with our previous observation that the sterile C. elegans mutant fer-1 is susceptible to S. enterica killing (Aballay et al., Curr. Biol. 10: 1539-1542, 2000), indicating that internal hatching of eggs was not the major factor involved in the killing of C. elegans hermaphrodites.

The Salmonella Type III Secretory System is Involved in C. elegans Killing

Several gram-negative animal and plant pathogens utilize the so-called type III secretion system to inject bacterial proteins into the cytosol of eukaryotic cells where the translocated proteins facilitate bacterial pathogenesis by specifically interfering with host cell signal transduction and other cellular processes (Galan et al. Science 284:1322-1328, 1999). A cluster of type III secretion system-related genes are located within Salmonella pathogenicity island 1 (SPI-1), including hilD and hilC which encode upstream regulatory factors required for the expression of the remaining SPI-1 genes. FIG. 1B shows that a S. enterica hilD and a double hilD/hilC mutant as well as a strain carrying a deletion of the entire SPI-1 cluster of genes exhibited reduced virulence compared to wild-type S. enterica, indicating that the type III secretion system is involved in C. elegans killing. Although these type III secretion system mutants exhibited reduced virulence in the C. elegans assay, they were still capable of efficiently killing C. elegans compared to E. coli OP50, indicating that virulence factors not encoded within SPI-1 are also involved in C. elegans killing.

To further investigate the role of SPI-1 encoded genes in C. elegans killing, the overexpression of the SPI-1 regulatory gene, hilA, was examined for whether it would enhance the rate of C. elegans killing. HilA, which functions directly downstream of HilD and HilC, is a transcriptional activator required for the expression of several SPI-1 genes including invF, which also encodes a transcriptional activator that in turn activates several SPI-1 genes, including sspC (Bajaj et al., Mol. Microbiol. 18:715-727, 1995; Lostroh et al., J. Bacteriol. 183:4876-4885, 2001). Consistent with the involvement of type III secretion system in C. elegans killing, S. enterica overexpressing HilA from plasmid pVV214 killed C. elegans more quickly than the wild type S. enterica control (FIG. 1C). The increased virulence caused by hilA overexpression could be prevented by an insertion mutation in invF that has a polar effect on genes encoding components of the type III secretion system (FIG. 1C).

Use of C. elegans to Screen a S. enterica TnphoA Library for Avirulent Mutants

Novel S. enterica virulence factors were identified by screening a TnphoA insertion library for mutants that failed to kill or exhibited attenuated killing of C. elegans. A total of 960 S. enterica TnphoA insertion mutants were individually screened for attenuated virulence using the glp-4 killing assay. Of these 960, 15 mutants, or 1.6%, consistently showed a lower rate of C. elegans killing when compared to wild-type S. enterica. These mutants were chosen for further analysis. All 15 mutants grew at the same rate as the wild-type strain in minimal (M9), rich (LB) or NG media, indicating that the attenuated pathogenicity phenotypes observed were not simply a result of growth defects. Moreover, DNA blot analysis showed that each of the mutant strains identified contained a single TnphoA insertion that conferred a similar avirulent phenotype after each of the TnphoA insertions was individually transduced into wild-type S. enterica strain SL1344 by phage P22 transduction. These results showed that the single TnphoA insertion in each of the 15 strains was responsible for the mutant phenotypes. In previously reported screens, P. aeruginosa virulence factors were identified using a similar screening method and 13 less virulent mutants were identified out of a total of 5,700 TnphoA insertion mutants screened, a yield of 0.23% (Tan et al., Proc. Natl. Acad. Sci. 96:2408-2113, 1999; Mahajan-Miklos et al., Cell 96:47-56, 1999). The number of mutants identified in the S. enterica screen was seven times greater, suggesting that C. elegans is a particularly efficient host for identifying Salmonella virulence factors.

As described herein, a PCR-based procedure was used to amplify DNA sequences adjacent to each of the TnphoA insertions in the 15 avirulent mutants. These mutants represented 1.6% of the 960 mutants screened using the C. elegans killing assay for mutants that failed to kill or exhibited attenuated virulence. The amplified sequences were subjected to DNA sequence analysis and the results, are summarized in Table 1.

TABLE 1

| | TnphoA | |
|---|---|---|
| Strain | insertion | Gene product description |
| 1E1 | hilA¹ | Transcriptional activator of SPI-1 invasion genes |
| 7D2 | hilD¹ | Transcriptional regulator that de-represses HilA |
| 9D5 | invH¹ | Component of the Type III secretion machinery |
| 2C4 | sptP^{1,2} | GTPase activating protein for host Cdc42 and Rac |
| 3E4 | srfH^{2*} | Virulence factor required for CEK |
| 3E11 | rhuM^{3*} | Virulence factor required for CEK |
| 6E5 | spi4-F^{4*} | Virulence factor required for CEK |
| 8F4 | pipA^{5*} | Required for enteropathogenesis and CEK |
| 2A6 | vsdA⁶ | Essential virulence factor in several systems |
| 4C4 | copB/repC⁶ | Plasmid copy number control |
| 2A4 | cstA^{*} | Carbon starvation protein in a fimbrial subunit |
| 3H7 | rfaL^{*} | O-antigen ligase required for CEK |
| 5F3 | leuO^{*} | Regulator involved in bacterial stringent response |
| 6D4 | gmhA^{*} | Essential for the expression of wild-type LOS |
| 7A2 | recB | Required for recombination |

¹genes located in Salmonella Pathogenicity Island-1,
²genes encoding effector proteins,
³genes located in Salmonella Pathogenicity Island-3,
⁴genes located in Salmonella Pathogenicity Island-4,
⁵genes located in Salmonella Pathogenicity Island-5,
⁶genes located in virulence plasmids,
^{*}genes not previously identified as virulence related.

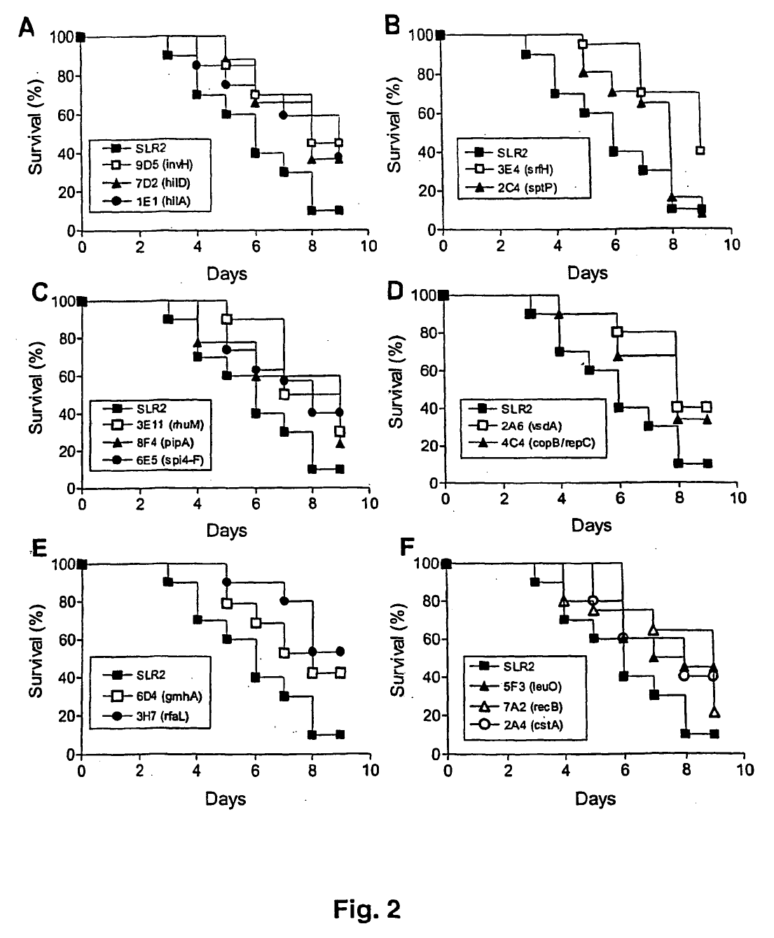

In addition, as shown in FIGS. 2A-2F, a detailed time course of C. elegans killing was determined for each of the 15 mutants.

Salmonella TnphoA Insertion Mutants in SPI-1 Genes and in Genes for Effector Proteins Identified by a C. elegans-Based Screen

Consistent with the results reported above, that type III secretion plays a role in S. enterica-induced killing, four Salmonella mutants with reduced virulence in C. elegans had TnphoA insertions in previously characterized virulence factors located in SPI-1 (Darwin et al., Clin. Microbiol. Rev. 12:405-428, 1999) (FIGS. 2A and 2B). InvH, one the genes targeted by TnphoA, is a component of the Type III secretion machinery, hilA and hilD, described above, and encodes regulatory proteins previously known to be required for complete virulence in animal models. Importantly, the SPI-1 sptP gene, identified in the TnphoA library, encodes an effector protein that is translocated into the host cytoplasm by the type III secretory sysem. The SPI-1 carboxyl-terminal domain has tyrosine phosphatase activity in vitro and displays amino acid sequence similarity to the Yersinia spp. tyrosine phosphatase YopH (Bolin et al., Mol. Microbiol. 2:237-245,1988; Kaniga et al., Mol. Microbiol 21:633-641,1996; Michiels et al., Microb. Pathog. 5:449-459, 1988). The amino-terminal domain of SptP has GTPase activating activity for Cdc42 and Rac and is similar to the bacterial cytotoxins YopE and ExoS (Forsberg, et al., J. Bacteriol. 172, 1547-1555, 1990; Fu et al., Nature, 401, 293-297, 1999; Michiels et al., Infect. Immun. 58, 2840-2849, 1990).

In addition to sptP, a second gene encoding a putative effector protein, srfH, was identified based on the reduced virulence presented in C. elegans by the strain 3E4 (FIG. 2B). srfH is located within a horizontally acquired region of the S. enterica chromosome which harbors the genes sspH1 and sspH2 that encode proteins translocated by the type III secretion system. srfH expression is strongly activated by the SPI-2 encoded transcription factor, SsrB, and based on sequence analysis is believed to be a type III secretion system-secreted effector (Worley et al., Mol. Microbiol. 36:749-761, 2000). A S. enterica mutant that contains a deletion of both sspH1 and sspH2 failed to cause a lethal infection in calves (Miao et al., Mol. Microbiol. 34:850-864, 1999). However, srfH has not previously been shown to be a virulence factor.

It is significant that the same translocated effectors appear to be involved in both mammalian and C. elegans pathogens. It was known that bacterial pathogens that infect evolutionarily disparate hosts (e.g., plants and mammals) employ common offensive strategies such as the translocation of virulence factors directly into host cells via a highly conserved type III secretion system. However, it was not known whether the translocated virulence factors (effector proteins) and their targets in different hosts were also conserved. These results with sptP and srfH suggest that C. elegans may be used to study the interaction between Salmonella effectors and their targets.

SrfH Expression in C. Elegans

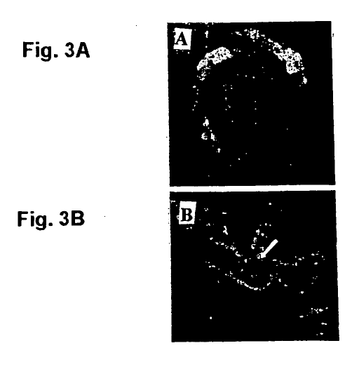

To characterize the interaction between the SrfH effector protein and its target in C. elegans, 2.5 kb of the 5′ UTR of the intestinal promoter kin-18 was fused to the srfH gene and the product obtained was subsequently fused to the GFP reporter sequence. Twenty animals were injected with this construct, and ten control animals were injected with a kin-18::GFP control construct (FIG. 3A). Of twenty animals injected with the kin-18::srfH::GFP construct, eight nematodes produced srfH::GFP embryonic expression in the F2 generation. The embryos produced by these nematodes failed to develop (FIG. 3B). Expression of the SrfH virulence factor was sufficient to alter host signaling pathways and block embryonic development. This discovery shows that C. elegans may be used to identify host signaling pathways targeted by Salmonella effector proteins. Such pathways may serve as therapeutic targets. In addition, a C. elegans screen for suppressors of the embryonic arrest phenotype may identify additional therapeutic targets.

Salmonella TnphoA Insertion Mutants in SPI-3, SPI-4, and SPI-5 Genes Identified by a C. elegans-Based Screen

In addition to the SPI-1 genes identified in the TnphoA library, three mutants with reduced virulence in C. elegans contained insertions in SPI-3, SPI-4, and SPI-5 genes (FIG. 2C). The SPI-3, SPI-4, and SPI-5 are horizontally acquired chromosomal clusters of pathogen-specific virulence genes identified as pathogenicity islands (Groisman et al., Cell 87:791-794, 1996; Hacker et al., Mol. Microbiol. 23:1089-1097, 1997). Mutant 3E11 contains a TnphoA insertion in the SPI-3 gene rhuM. This pathogenicity island harbors mgtC, a Salmonella-specific gene that is required for intramacrophage survival, virulence in mice, and growth in low-Mg²⁺ media (Blanc-Potard et al., J. Bacteriol. 181:998-1004, 1999). However, two other SPI-3 encoded genes, misL and marT, which have sequence similarity to known virulence factors, could not be shown to play a role in survival within macrophages, invasion of epithelial cells, or in virulence in a BALB/c typhoid mouse model (Blanc-Potard et al., J. Bacteriol. 181:998-1004, 1999). rhuM had not previously been shown to be a virulence-related factor. Thus, besides mgtC, our results show that there may be additional virulence-related genes encoded within SPI-3.

Mutant 6E5 contains a TnphoA insertion in the SPI-4 gene spi4-F, which shares significant homology with a hypothetical protein of Acinetobacter calcoaceticus (Wong et al., Infect. Immun. 66:3365-3371, 1998), but like rhuM and pipA, has not previously been shown to encode a virulence factor. SPI-4 contains 18 putative open reading frames encoding proteins that have significant homology with proteins involved in toxin secretion. These proteins have significant homology with hypothetical proteins from Synechocystis sp, and novel proteins (Wong et al., Infect. Immun. 66:3365-3371, 1998).

Mutant 8F4 contains a TnphoA insertion in the SPI-5 pipA gene. The SPI-5 genes pipD, sopB, pipB, and pipA have been shown to be involved in Salmonella enteropathogenesis, but they are not required for the virulent phenotype in a BALB/c typhoid mouse model (Wood et al., Mol. Microbiol. 29:883-891, 1998).

The Salmonella Virulence Plasmid is Required for Complete Virulence in C. elegans

The virulence properties of various Salmonella serovars depends on the presence of large plasmids of variable size, ranging from 50 to 94 kb. All of these virulence plasmids contain a highly conserved 8-kb region that contains the spv locus, which is required for maximum virulence in animal models (Gulig et al., Mol. Microbiol. 7:825-830, 1993; Gulig et al., Infect. Immun. 61:504-511, 1993). TnphoA mutant 3E11 contains an insertion in spvA, which was previously shown to be essential for virulence (Krause et al., J. Bacteriol. 173:5754-5762, 1991). Mutant 4C4 contains an insertion in copB/copC, which is also located in the Salmonella virulence plasmid, but outside of the spv locus. The copB/repC gene is known to be involved in plasmid copy number control (Haneda et al., Infect. Immun. 69:2612-2620, 2001). FIG. 2D shows that Salmonella virulence plasmids play an important role in C. elegans killing.

Salmonella Mutants in Which Normal LPS Expression is Disrupted Present a Reduced Virulence in C. elegans

It is well known that lipopolysaccharide (LPS) plays a critical role in inflammation elicited by gram-negative pathogens and in the pathogenesis of the diseases caused by these organisms. The reduced virulence of the mutant 6D4 (FIG. 2E) is attributed to a TnphoA insertion in the Salmonella homologue of E. coli gmhA, which encodes a phosphoheptose isomerase essential for the expression of wild-type LPS. Mutant 3H7 has TnphoA inserted in rfaL, which encodes O-antigen ligase that is required for wild type biosynthesis of LPS.

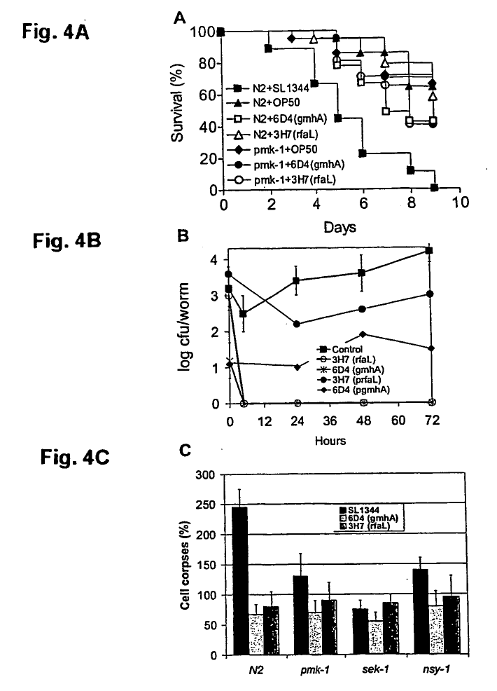

We found that 6D4 and 3H7 (FIG. 4A) failed to establish a persistent infection in the C. elegans intestine. Five hours after C. elegans were shifted from Salmonella to E. coli, the 6D4 and 3H7 mutants were no longer detected (FIG. 4B). In contrast, Salmonella containing a TnphoA control insertion in a gene unrelated to pathogenesis persisted and proliferated in the C. elegans intestine for at least three days. 6D4 and 3H7's failure to establish a persistent infection was restored when wild-type gmhA or rfaL genes were expressed under the control of the constitutive lacZ promoter in strains 6D4 and 3H7, respectively. This expression also restored the virulence of the mutant strains, although the virulence level of the lacZ::gmhA expressing 6D4 strain was lower than that observed for wild-type Salmonella. This difference is likely due to a polar effect on a downstream gene present in an operon with gmha.

LPS produced by gmhA and rfaL mutants contains lipid A, the component of LPS that triggers a variety of host defense mechanisms in mammals, including programmed cell death (Medzhitov Cell 91:295-298, 1997; Navarre et al., Cell Microbiol 2:265-273, 2000). We have previously shown that Salmonella enterica serovars cause a persistent infection in the C. elegans intestine (Aballay et al., Curr. Biol. 10:1539-1542, 2000; Labrousse Curr. Biol. 10:1543-1545, 2000) that triggers gonadal programmed cell death. We found that 6D4 (gmhA) or 3H7 (rfaL) mutants failed to induce Salmonella-induced germ-line cell death (FIG. 4C). This result suggested that intact LPS is required for induction of Salmonella-induced gonadal cell death. The other 13 TnphoA mutants tested all elicited the same level of programmed cell death as wild-type Salmonella. This result suggested that intact LPS is required for the Salmonella-induced gonadal programmed cell death. Moreover, the level of gonadal programmed cell death in pmk-1 RNAi worms feeding on the gmhA or rfaL mutants was equal to that observed in wild-type worms feeding on E. coli. This suggested that basal level of gonadal programmed cell death observed when worms are fed E. coli is not triggered by bacterial LPS. Consistent with these results, the same basal level of gonadal programmed cell death was observed in worms fed E. coli deficient in LPS biosynthesis or non-pathogenic Gram positive bacteria. Importantly, both 6D4 (gmhA) and 3H7 (rfaL) accumulated to high titers in the C. elegans intestinal lumen (FIG. 4B), although they failed to cause a persistent infection. Thus, this failure is unlikely to be caused by low levels of bacterial accumulation. Rather, intact LPS is likely required for Salmonella to establish a persistent infection. Intact LPS, for example, may be required for Salmonella to adhere to receptors present on C. elegans intestinal cells. Consistent with this conclusion, we found that virulence in 6D4 (gmhA) and 3H7 (rfaL) mutants could not be restored by exogenous application of purified S. enterica LPS.

C. elegans and S. enterica Factors that Affect Salmonella-Induced Programmed Cell Death

In mammals, p38 MAPK is involved in mediating the innate immune response to bacterial LPS, and p38 activation results in programmed cell death in mammalian cells. C. elegans having mutations in MAPK pathway components have enhanced susceptibility to pathogens, and the MAPK pathway likely functions in C. elegans innate immunity. To determine whether the p38 MAPK signaling pathway plays a role in gonadal programmed cell death in C. elegans, we looked at Salmonella-induced gonadal programmed cell death in C. elegans having MAPK pathway mutations.

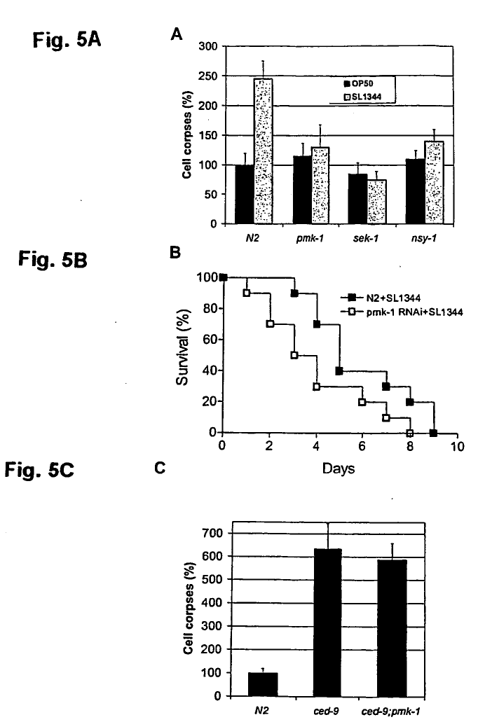

PMK-1, SEK-1 and NSY-1 are the C. elegans homologues of p38, MAPK-kinase, and MAPK-kinase-kinase, respectively (Sagasti et al., Cell 2001; 105:221-232; Tanaka-Hino et al., EMBO Rep 3:56-62, 2002). When grown on Salmonella, wild-type worms have increased levels of gonadal programmed cell death. Salmonella failed however to induce gonadal programmed cell death in sek-1 or nsy-1 mutant worms, or in worms in which pmk-1 had been inactivated by RNA interference (RNAi) (FIG. 5A). These results demonstrated that Salmonella-induced gonadal programmed cell death is PMK-1-dependent, but that the “basal” level of programmed cell death observed in C. elegans feeding on E. coli is PMK-1-independent. This is consistent with our previous results, which showed that worms in which Salmonella-induced cell death is blocked are hypersusceptible to Salmonella-induced killing.

RNAi pmk-1 worms were also more susceptible to Salmonella-induced killing than wild-type worms (FIG. 5B), although the life span of RNAi pmk-1 worms and wild-type worms on E. coli was comparable (FIG. 4A). Thus, the shortened life span of the RNAi pmk-1 worms feeding on Salmonella is not a consequence of pmk-1 worms being sickly.

CED-9 Lies Downstream of PMK-1

To verify that the CED cell death pathway is acting downstream of pmk-1, we used worms carrying a loss-of-function mutation in the ced-9 gene. C. elegans ced-9 encodes a negative regulator of programmed cell death (Hengartner et al., Nature 356:494-499, 1992). Worms having a ced-9 gain of function mutation are cell death defective (ced). We have previously shown that Salmonella-induced gonadal programmed cell death is blocked in ced-9 gain of function mutants, indicating that Salmonella-induced programmed cell death is CED-9-dependent. PMK-1 inactivation by RNAi in a ced-9 loss-of-function mutant did not reduce the high level of spontaneous gonadal cell death (FIG. 5C). These results suggested that Salmonella-induced gonadal cell death is ced-9 dependent, and that Salmonella induced cell death is downstream of pmk-1.

Salmonella-Induced Programmed Cell Death and PMK-1 Activation are not Dependent on C. elegans Toll-Like Receptor

In response to pathogen-associated molecular patterns (AMPs), vertebrate Toll-like receptors (TLR) activate several signaling pathways, including the p38 MAPK pathway (Aderem et al., Nature 406:782-787, 2000). The C. elegans genome appears to encode a single TLR, TOL-1, as well as single copies of TRF-1, PIK-1, and IKB-1, homologues of the mammalian downstream signal transduction components TRAF1, IRAK, and IκB, respectively (Pujol et al, Curr Biol 11:809-821, 2001). The C. elegans genome does not appear to encode Rel-like transcription factors and it is not known whether C. elegans responds to bacterial-encoded PAMPs. Indeed, C. elegans TLR-associated signaling components appear to be associated with the avoidance of potential pathogens (Pujol et al, Curr Biol 11:809-821, 2001), rather than with the activation of host defense responses.

To determine whether the TOLL-receptor pathway is required for PMK-1-dependent Salmonella-induced programmed cell death, C. elegans tol-1, trf-1, and pik-1 deletion mutants were fed Salmonella, and gonadal cell death was monitored. The level of Salmonella-induced programmed cell death in the deletion mutants was comparable to the level observed in wild-type C. elegans. This indicated that TOL-1 is not the C. elegans receptor that senses the stimulatory signal that activates PMK-1. In addition, it demonstrated that the presumptive C. elegans TOLL-pathway is not required for Salmonella-induced programmed cell death. These results further indicated that one or more receptors, other than TOLL-like receptors, are acting upstream of the MAPK-dependant CED cell death pathway in C. elegans. Consistent with this hypothesis, wild-type levels of activated PMK-1 were observed in tol-1 mutants.

In Drosophila, the Toll signaling pathway appears to mediate the activation of Dredd, a homologue of the C. elegans caspase, CED-3, and it has been suggested that the Toll-induced pathway of caspase activation is the evolutionary ancestor of the death receptor-mediated pathway for apoptosis induction in mammals (Horng et al., Proc. Natl. Acad. Sci. 98:12654-12658, 2001). Our finding, that Salmonella-induced programmed cell death and PMK-1 activation are not dependent on C. elegans Toll-like receptor, suggested that another pathway, distinct from the Toll signaling pathway, is the ancestor of the mammalian death-receptor pathway. Moreover, given the fact that C. elegans does not appear to contain any Rel-like family transcription factors, it is likely that both the upstream and downstream components of the MAPK cascade involved in the orchestration of host defense responses against Gram negative pathogens are not conserved between mammals and C. elegans. This suggested that the p38 MAPK cascade is likely the most ancient conserved feature of the host defense response against Gram negative pathogens in metazoans.

Although the results described above, as well as the data published by Pujol et al. (Curr Biol 11:809-821, 2001), suggested that the C. elegans Toll-like signal transduction pathway is not involved in the recognition of bacterial PAMPs such as LPS, it is likely that C. elegans is nevertheless responding to Salmonella-encoded signals in the activation of gonadal programmed cell death.

We had previously observed that S. enterica having mutations in the PhoP/PhoQ regulatory system are highly attenuated in virulence, failed to induce programmed cell death, and failed to establish a persistent infection in the C. elegans intestine (Aballay et al., Proc. Natl. Acad. Sci. 98:2735-2739, 2001). Mutations in the S. enterica phoP/phoQ genes are known to affect a variety of virulence-related factors, including the synthesis of LPS (Ernst et al., Microbes Infect. 3:1327-1334, 2001). We tested the effects of LPS-synthesis avirulent mutants gmhA and rfaL on RNAi pmk-1 worms, and found that survival of RNAi pmk-1 worms on the mutants was comparable to that exhibited by wild-type C. elegans (FIG. 4A). This showed that the avirulent phenotype of the LPS mutants is epistatic to the hypersusceptibility phenotype of the RNAi pmk-1 worms.

By showing that programmed cell death is downstream of the p38-signaling cascade, this work linked PMK-1 signaling with programmed cell death. This work also showed that Salmonella-activated programmed cell death is dependent on intact LPS. Without being tied to a particular theory, it may be that LPS signaling is also mediated by PMK-1 signaling. If this were the case, then both the mammalian and C. elegans innate immune responses utilize p38, not only as a key innate immunity signaling component, but also to mediate response to LPS as a PAMP. Initial attempts to show that purified LPS activated p38, or that Salmonella or E. coli LPS mutants failed to activate p38 were inconclusive, most likely due to technical limitations. LPS, for example, may lead to p38 (PMK-1) activation in only a subset of C. elegans cells that cannot be detected in whole animal lysates. It is also possible that LPS activates a signaling pathway in parallel to the p38 pathway that leads to programmed cell death. Another possibility is that two independent signals, one of which is LPS, are required to activate the programmed cell death pathway, thereby allowing C. elegans to discriminate between Gram negative bacteria in general and Gram negative pathogens. A similar situation may exist in Drosophila, where it has been suggested that a peptidoglycan recognition protein (PGRP-LC) binds both peptidoglycan and LPS, or cooperates with a parallel signaling pathway involving other pattern recognition receptors that bind LPS directly (Navarre et al., Cell Microbiol 2:265-273, 2000). Finally, the component of LPS recognized by PAMP receptors in mammals is lipid A, a structure that is intact in both 6D4 (gmhA) and 3H7 (rfaL) mutants. Thus, our results suggested that C. elegans recognizes modifications in the Salmonella LPS outer core. Interestingly, S. enteritidis 1047, which synthesizes a different LPS outer core than S. enterica, also elicited programmed cell death levels comparable to those observed in C. elegans infected with S. enterica.

These results demonstrate that an important feature of innate immune signaling pathways, the activation of programmed cell death downstream of a p38 MAPK signaling cascade, is conserved between nematodes and mammals. Thus, this work provided important new insights into the evolutionary origins of innate immunity. This work also demonstrated that C. elegans may respond to a component of LPS. Whether this component is a highly conserved PAMP remains to be determined. In any case, it appears that C. elegans recognizes a different component of LPS than is recognized by mammalian TLRs. Finally, the Salmonella LPS signal that activated the C. elegans programmed cell death response apparently functioned independent of a TOLL-like pathway. It is likely that one of the most prominent features of insect and mammalian innate immunity, the involvement of TLRs in LPS signaling, may not be conserved in nematodes.

In sum, we have identified both C. elegans and S. enterica factors that affect Salmonella-induced programmed cell death. Wild-type Salmonella lipopolysaccharide (LPS) was required for Salmonella-induced gonadal programmed cell death, as well as for the persistence of Salmonella in the C. elegans intestine. Salmonella-induced programmed cell death also required the C. elegans homologue of the mammalian p38 mitogen-activated protein kinase (MAPK), which is encoded by the pmk-1 gene. Inactivation of pmk-1 by RNAi blocked Salmonella-induced programmed cell death, and epistasis analysis showed that ced-9 lies downstream of pmk-1. These results suggested that genes involved in innate immunity could be identified by screening for C. elegans mutants having reduced levels of gonadal programmed cell death on Salmonella.

TnphoA Insertions in Various Chromosomal Regions of S. enterica Reduce its Virulence in C. elegans

FIG. 2F shows the reduced virulence of the mutant 5F3. Since TnphoA is inserted 56 bases upstream of an open reading frame with significant sequence similarity to the E. coli transcriptional regulator LeuO, the reduced virulence in C. elegans is most probably due to the disruption of the promoter of the Salmonella leuO gene. In E. coli, leuO is required for resumption of growth after a two hour growth arrest caused by starvation for branched-chain amino acids, suggesting a role for leuO in the bacterial stringent response (Majumder et al., J. Biol. Chem. 276:19046-19051, 2001).

The TnphoA insertion in mutant 2A4 is located in a fimbrial subunit, 100 bases upstream of a homolog of the E. coli cstA gene (FIG. 2F). This is consistent with the observation that the ability of S. enterica and several other pathogens to bind to host cells is a critical step in pathogenesis.

The reduction in virulence of the mutant 7A2 (FIG. 2F) is due to a TnphoA insertion in recB. It is known that recombination deficient mutants of several pathogen species are generally less virulent, and that Salmonella recombination deficient mutants were attenuated in mice (Buchmeier et al., Mol. Microbiol. 7:933-960, 1993).

S. enterica Mutants are Defective in Both C. elegans Killing and Induction of Polymorphonuclear Leukocyte Migration

Eight of the 15 mutants identified in the screen contained TnphoA insertions in genes that had not been previously shown to be involved in virulence, or for which a role in virulence had been presumed, but not clearly demonstrated (Table 1). To determine whether these genes are also involved in mammalian pathogenesis, the corresponding TnphoA insertion mutants were tested for their ability to invade epithelial cells and induce polymorphonuclear leukocytes migration in mammalian cell culture. It is known that interaction between Salmonella and intestinal epithelial cells can induce epithelial cells to release signaling factors that direct the transepithelial migration of polymorphonuclear leukocytes (Eckmann et al., Infect. Immun. 61:4569-4574, 1993; McCormick et al., J. Cell Biol. 123:895-907, 1993; McCormick et al., Infect. Immun. 63:2302-2309, 1995; McCormick et al., J. Immunol. 160:455-466, 1998).

Table 2 shows the ability of eight TnphoA mutants to promote the invasion of epithelial cells and the transepithelial migration of polymorphonuclear leukocytes. Interestingly, all eight of the mutants showed a reduced ability to promote transepithelial migration of polymorphonuclear leukocytes when compared to wild-type Salmonella. In contrast, at least three random TnphoA insertion mutants that did not affect C. elegans killing were not distinguishable from wild-type SL1344 in either the epithelial cell invasion or the polymorphonuclear leukocyte transepithelial migration assays (data not shown). Although the rhuM mutant exhibited the least reduction in the polymorphonuclear leukocyte transepithelial migration assay of the 8 mutants tested, it did show a significant decrease in epithelial cell invasion. The rest of the mutants tested showed significant reductions in both epithelial invasion and transepithelial migration, with the exception of the leuO mutant, which exhibited wild-type levels of epithelial invasion, but highly reduced transepithelial migration of polymorphonuclear leukocytes.

It is interesting that with the exception of rhuM, all eight of the mutants tested elicited a significant reduction in polymorphonuclear migration (Table 2). These results suggest that the C. elegans model can be readily used to identify novel Salmonella virulence factors capable of altering host-defense response signaling pathways in cells that are not in direct contact with Salmonella.

Epithelial Invasion and Polymorphonuclear Leukocyte Transmigration Phenotypes of Salmonella enterica TnphoA Insertion Mutants with Reduced Virulence in C. elegans.

The physiologically directed (baso-lateral-to-apical) polymorphonuclear leukocyte transepithelial migration assay using cell culture inserts of inverted T84 monolayers was carried out as already described (Eckmann et al., Infect. Immun. 61:4569-4574, 1993; McCormick et al., J. Cell Biol. 123:895-907, 1993; McCormick et al., Infect. Immun. 63:2302-2309, 1995; McCormick et al., J. Immunol. 160:455-466, 1998). Human polymorphonuclear leukocytes were isolated from normal volunteers. The S. enterica SL1344 and VV341 (hilA::kan-339) were used as controls. The C. elegans killing was calculated 8 days after infection. Data are expressed as percentage of wild-type response. Mouse killing was calculated 17 days after infection. Data are expressed as percentage of wild-type response.

TABLE 2

| | | PMN | Epithelial | C. elegans |
|---|---|---|---|---|
| | | Transmigration | Cell Invasion | Killing⁶ |
| | TnphoA | (% of wild | (% of wild | (% of wild |
| Strain | Insertion | type response) | type response) | type response) |
| SLR2 | — | 100 | 100 | 100 |
| VV341 | hil4¹ | 04 | 83 | ND |
| 2A4 | cstA | 39 | 54 | 67 |
| 3E4 | srfH² | 50 | 46 | 33 |
| 3E11 | rhuM³ | 81 | 56 | 55 |
| 3H7 | rfaL | 01 | 04 | 50 |
| 5F3 | leuO | 37 | 107 | 67 |
| 6D4 | gmhA | 31 | 12 | 67 |
| 6E5 | spi4-F⁴ | 15 | 61 | 67 |
| 8F4 | pipA⁵ | 11 | 63 | 45 |

ND, not determined.
¹Genes located in Salmonella Pathogenicity Island-1. S. enterica strain VV341 (hilA::kan-339) was used as a control.
²genes encoding effector proteins.
³genes located in Salmonella Pathogenicity Island-3.
⁴genes located in Salmonella Pathogenicity Island-4.
⁵genes located in Salmonella Pathogenicity Island-5.
⁶The C. elegans killing was calculated 8 days after the infection.

Some S. enterica Mutants Defective in C. elegans Killing are Avirulent in a BALB/c Typhoid Mouse Model

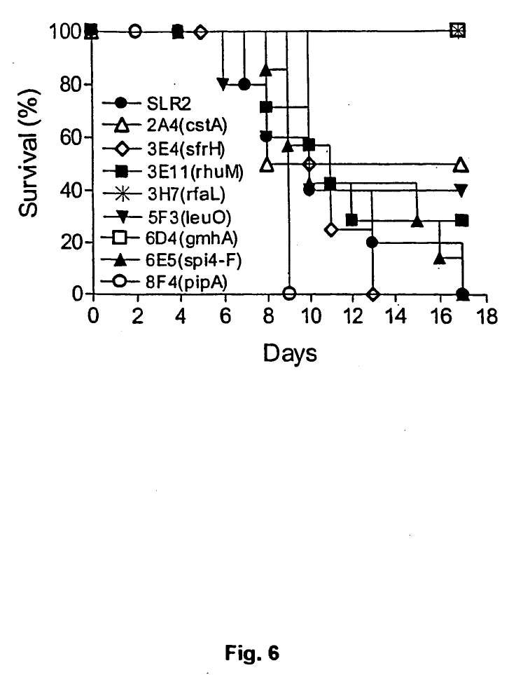

Many Salmonella genes required for full virulence in mice have been identified, but only a few of these have been shown to be necessary for inducing migration of polymorphonuclear leukocytes into the intestinal mucosa and lumen from the underlying microvasculature, a key virulence determinant underlying the development of Salmonella-elicited enteritis (McCormick et al., Infect. Immun. 63:2302-2309, 1995). Likewise, at least some of the virulence factors affecting enteritis do not appear to be required for infection of systemic sites in mice, suggesting that a subset of genes influences different aspects of Salmonella pathogenesis. Therefore, the virulence of the bacterial mutants for which the polymorphonuclear leukocyte transepithelial migration was studied were tested in a typhoid mouse model (Monack et al., J. Exp. Med., 192:249-258, 2000; Pepe et al., Infect. Immun. 63:4837-4848 1995). Two of these mutants, 3H7 (rfaL) and 6D4 (gmhA) failed to kill any of the inoculated mice (FIG. 6). Two additional mutants, 2A4 (cstA) and 5F3 (leuO), may exhibit some reduction in virulence 17 days after infection when compared to wild type (FIG. 6).

In this work both forward and reverse genetic analysis have been used to identify bacterial virulence factors that are required for C. elegans killing as well as mammalian pathogenesis, thereby validating the use of C. elegans as a model host for Salmonellosis. Two Salmonella effector proteins known to be translocated into mammalian cells by the type III secretion system were also shown to be required for virulence in C. elegans. This result is particularly interesting because it suggests that virulence factor targets in the host are broadly conserved. Consistent with this hypothesis, the expression of the catalytic subunit of pertussis toxin in C. elegans produced phenotypes almost identical to those of a null mutation in the nematode gene encoding G(o/i)alpha, the mammalian target of the toxin (Darby et al., Infect. Immun. 69:6271-6275, 2001). Moreover, eight out of fifteen mutants isolated in our screen correspond to disruptions in genes that have not been definitively related to virulence before. Seven out of these latter eight mutants exhibited reduced polymorphonuclear transepithelial migration, indicating an important correlation between the C. elegans and polymorphonuclear transmigration assay. Finally, the gmhA and rfaL genes were identified not only as major components of Salmonella-elicited polymorphonuclear transepithelial migration, but also as major components of Salmonella virulence in mice.

Our results show that there is a significant overlap between Salmonella virulence factors required for human and nematode pathogenesis, and that the screening method described represents a tool with which to dissect the genetic basis of the Salmonella interaction with its hosts. The screen should be applicable to study the entire genome of this pathogen.

Described below are detailed materials and methods relating to the above-described identification of Salmonella enterica virulence factors.

Bacterial Strains and Growth Conditions

E. coli OP50 (Brenner, Genetics 77:71-94, 1974) or S. enterica SL1344 (Wray et al., Res. Vet. Sci. 25:139-143, 1978) have been described. S. enterica SLR2 is a spontaneous rifampicin-resistant derivative of SL1344. Derivatives of S. enterica SL1344, LM399 (hilD::kan-1) (Schechter et al., Mol. Microbiol. 32:629-642 1999), LS666 (hilD::kan; hilC::cam), and SD11 (ΔSPI-1) (Schechter et al., Mol. Microbiol., 32:629-642, 1999) have been described and were kindly provided by C. Lee, Harvard Medical School. S. enterica SL1344 and SL1344 invF::Tn5lacZY overexpressing hilA from plasmid pVV214 have been described (Bajaj et al., Mol. Microbiol. 18:715-727, 1995). Bacterial cultures were grown in Luria-Bertani (LB) broth or M63 (Ausubel et al., Current Protocol in Molecular Biology, New York: John Wiley and Sons, 1997) minimal medium with appropriate antibiotics at 37° C. Media were solidified with 1.5% Bacto Agar (Difco). The following antibiotic concentrations (per milliliter) were used in this study; kanamycin, 200 μg/ml and rifampicin 100 μg/ml for S. enterica; kanamycin, 50 μg/ml for E. coli. Antibiotics were purchased from Sigma (St. Louis, Mo.). Bacterial lawns used for C. elegans killing assays were prepared by spreading 10 μl of an overnight culture of the bacterial strains on modified NG agar medium (0.35% instead of 0.25% peptone) in 3.5 cm diameter plates. Plates were incubated at 37° C. for twelve hours and then allowed to equilibrate to room temperature for three hours before seeding them with worms.

DNA Manipulations

Procedures for general DNA manipulations, including extraction of plasmid DNA, restriction enzyme digestions and agarose gel electrophoresis have been described previously by Ausubel et al. (supra).

C. elegans Strains and Killing Assay

C. elegans wild type N2 animals were maintained as hermaphrodites at 20° C., grown on modified NG agar plates (0.35% instead of 0.25% peptone) and fed with E. coli strain OP50 as previously described (Brenner, Genetics 77:71-94, 1974). C. elegans glp-4 animals were maintained as hermaphrodites at 15° C. To produce sterile glp-4 animals, gravid glp-4 adult worms were grown at 15° C. for one day and then the plates containing L1 animals were transferred to 25° C. for two days. For the killing assay, ten N2 or glp-4 worms were placed on a bacterial lawn and the plates were incubated at 25° C. Each independent assay was carried out in duplicate. When using N2 worms, adults were transferred daily to fresh plates during the reproductive period and thereafter were transferred approximately every five days. Worm mortality was scored over time, and a worm was considered dead when it failed to respond to touch. The glp-4 mutant used was obtained from the Caenorhabditis Genetics Center, University of Minnesota, St. Paul, Minn.

C. elegans Strains and Cell Death Assays

sek-1 and nsy-1 were identified in our laboratory (Kim et al., Science 297:623-626, 2002). RNAi pmk-1 worms were obtained by growing the nematodes as described by Timmons and Fraser (Timmons et al., Nature 395:854 1998; Fraser et al., Nature 408:325-330, 2000). Progeny of these worms were transferred to modified NG agar medium containing 1:1 mixtures of S. enterica and E. coli containing an L4440-derived vector with sequence specific to pmk-1.

Screen for bacterial mutants deficient in C. elegans killing S. enterica SLR2 mutant libraries were generated using the TnphoA transposon (Manoil et al., Proc. Natl. Acad. Sci. 82:8129-33, 1985) using a modification of published protocols (Simons et al., Mol. Plant. Microbe. Interact. 9:600-607, 1996). S. enterica SLR2 (recipient) and E. coli strain SM10 λpir (Taylor et al., J. Bacteriol. 171:1870-8, 1989) (donor) were grown in LB to late log phase (A₆₀₀=0.6-0.8). Five hundred microliters of each donor and recipient were mixed in an Eppendorf tube. The cells were pelleted in a microfuge, washed with sterile 10 mM MgSO₄, and resuspended in 250 μl of LB. Ten aliquots, of 25 μl each, were spotted on an LB plate and incubated at 37° C. for 6-8 hours. After incubation, the cells in each of the spots were scraped from the LB plate, resuspended in 1.0 ml of LB, and twenty 50 μl aliquots from each of the ten 1.0 ml cultures were plated on LB plates supplemented with kanamycin (to select for the TnphoA mutants) and rifampicin (to select against growth of the E. coli donor). The resulting transposon mutants were screened as described below. Individual SLR2::TnphoA transposant strains were grown overnight at 37° C. in 200 μl LB liquid broth in 96-well microtiter plates. Bacterial lawns used for C. elegans killing assays were prepared by spreading 10 μl of the bacterial transposant strains on modified NG agar medium (0.35% instead of 0.25% peptone) in 24-well plates and incubated overnight at 37° C. Plates were allowed to equilibrate to room temperature for three hours before seeding seven glp-4 worms per well. Plates were incubated at 25° C. and examined for more than five live worms after seven days. Putative nonpathogenic or attenuated mutants identified in the preliminary screen were retested, and subjected to the C. elegans killing assay described above to determine the kinetics of C. elegans killing.

Molecular Analysis of TnphoA Mutants

Genomic DNA isolated from S. enterica SL1344 and TnphoA mutants Ausubel et al. (Current Protocols in Molecular Biology, Wiley Interscience, New York, 1997) was digested with the restriction endonucleases XhoI and PstI. DNA blots were prepared as previously described (Ausubel et al. Current Protocols in Molecular Biology, Wiley Interscience, New York, 1997) and hybridized with a TnphoA fragment generated using primers 5′-GCAGAAACGTTGGGATTGC-3′ (SEQ ID NO:15) and 5′-ACTGGAGGATTGTACCATTGCA-3′ (SEQ ID NO:16) which were labeled according to standard protocols (Ausubel et al. (supra). Sequences flanking the 3′ transposon ends of the TnphoA mutants were amplified using arbitrarily-primed PCR(O'Toole et al., Mol. Microbiol. 28:449-461, 1998) and the PCR products ranging from 200 to 400 bp were sequenced. The sequence information was compared with the nonredundant databases at the National Center for Biotechnology Information.

Construction of rfaL and gmhA Complementing Clones

The rfaL and gmhA genes were amplified from S. enterica SL1344 chromosomal DNA using primers (rfaL1) 5′-TCGTATCGGTTGATACCGGC (SEQ ID NO:19); (rfaL2) 5′-GAACCTATGTCGAGCGACAG (SEQ ID NO:20) and (gmhA1) 5′-CTGACCACTTGTGATGATTA (SEQ ID NO:21), (gmhA2) 5′-AGCAGATATCCGTCGGCACA (SEQ ID NO:22), respectively. The amplified products were first cloned into the pCR 2.1-TOPO cloning vector (Invitrogen). Subsequently, a 1.68 kb EcORI rfaL-containing fragment, which contains only the an intact rfaL open reading frame, and a 1.07 kb EcORI gmhA-containing fragment, which contains only the an intact gmhA open reading frame, were subcloned into the corresponding sites of pUCP19 and pUCP18, respectively, such that expression of both genes was driven by the plasmid lacZ promoter.

Cell Culture

T84 intestinal epithelial cells (passages 45 to 65) were grown in a 1:1 mixture of Dulbecco-Vogt modified Eagles medium and Ham's F-12 medium supplemented with 15 mM Hepes buffer (pH 7.5), 14 mM NaHCO3, 40 mg/liter penicillin, 8 mg/liter ampicillin, 90 mg/liter streptomycin, and 5% newborn calf serum. Polarized monolayers of T84 cells were formed and maintained on 0.33-cm2 ring-supported collagen-coated polycarbonate filters (Costar Corp., Cambridge, Mass.), as previously described (Dharmsathaphorn et al. Methods Enzymol. 192:354-389, 1990) with recently detailed modifications (Madara et al., J. Clin. Invest. 89:1938-1944, 1992). T84 cell monolayers reached a steady-state resistance four to six days after plating with some variability largely related to cell passage number. For clarity, this polycarbonate filter with the attached monolayer of T84 cells and matrix is referred to as “cell culture inserts”. Cell culture inserts were utilized for bacterial invasion and polymorphonuclear leukocyte transmigration assays five to fourteen days after plating, as described previously (Madara et al., J. Clin. Invest. 89:1938-1944, 1992). Cell culture inserts received one weekly feeding following initial plating. Cell culture inserts of inverted monolayers, used to study transmigration of polymorphonuclear in the physiological basolateral-to-apical direction, were constructed as previously described (Madara et al., J. Clin. Invest. 89:1938-1944, 1992; Nash et al., J. Clin. Invest. 87:1474-1477, 1991; Parkos et al., J. Cell. Biol. 117:757-764, 1991).

Growth of Bacteria for Assays Using Cell Culture Inserts

Non-agitated microaerophilic bacterial cultures were prepared by inoculating 10 ml of LB broth with 0.01 ml of a stationary-phase culture, followed by overnight incubation (approximately 18 hours) at 37° C. Bacteria from such cultures were in the late logarithmic phase of growth and correlated with 5-7×10⁸ CFU/ml (colony forming units per ml). Routinely, CFU were determined by diluting and plating onto MacConkey agar medium (Difco) or L agar, as previously detailed (McCormick et al., J. Cell Biol. 123:895-907, 1993; McCormick et al., Infect. Immun. 63:2302-2309, 1995). Ampicillin (50 μg/ml) was added to bacterial culture media when necessary.

S. enterica Cell-Association and Invasion Assays

Infection of T84 monolayers was performed by the method described previously (McCormick et al., J. Cell Biol. 123:895-907, 1993; McCormick et al., Infect. Immun. 63:2302-2309, 1995). Briefly, cell culture inserts of T84 cells were prepared as described above. After confluency was established (usually five days after plating), the cell culture inserts were lifted from the wells, drained of media by inverting, and gently washed by immersion in a beaker containing Hank's Buffered Salt Solution, plus Ca²⁺ and Mg²⁺, (Sigma Chem. Co., St. Louis, Mo.) with 10 mM HEPES, pH 7.4 (hereafter termed HBSS+). The cell culture inserts were placed in a new 24-well tissue culture plate with 1.0 ml HBSS(+) in the lower (basolateral) well and 0.05 ml HBSS(+) added to the upper (apical) well. After a thirty minute equilibration, 10 μl of bacteria washed and resuspended in HBSS(+) was added apically to each cell culture insert. This represents an inoculation ratio of approximately twenty bacteria/epithelial cell. S. enterica attachment to and entry into T84 intestinal epithelial cells was assessed after one hour. Cell-associated Salmonella representing populations of bacteria attached to and/or internalized into the T84 monolayers were released from the monolayer by incubation with 0.1 ml of 1% Triton X-100 (Sigma Chemical Corp.). Internalized bacteria were those obtained from lysis of the epithelial cells with 1% Triton X-100, 90 minutes after the addition of gentamicin (50 μg/ml). Preliminary gentamicin dose-response studies defined the conditions required to achieve bacteriocidal effects on the strain used. For both cell-associated and internalized bacteria, 0.9 ml LB broth was added after the Triton X-100 incubation and each sample was vigorously mixed and quantified by plating for CFU on MacConkey agar medium. To determine the number of attached Salmonella, the number of cell-internalized CFU was subtracted from the number of cell-associated CFU (since cell-associated values represent both attached and internalized bacteria).

Polymorphonuclear Leukocyte Transepithelial Migration Assay

The physiologically directed (basolateral-to-apical) polymorphonuclear leukocyte transepithelial migration assay using cell culture inserts of inverted T84 monolayers has been previously described (Parkos et al., J. Cell Biol. 88:1605-1612, 1991). Human polymorphonuclear leukocytes were isolated from normal volunteers, as described elsewhere (Henson et al., J. Clin. Invest. 56:1053-1061, 1975; Parkos et al., J. Cell Biol. 88:1605-1612, 1991). Briefly, polymorphonuclear leukocytes are routinely isolated from anti-coagulated whole blood (150 to 500 ml) collected by venipuncture from normal donors of both sexes. The buffy coat was obtained via a 400×g spin at room temperature. Plasma and mononuclear cells were removed by aspiration, and the majority of erythrocytes were removed using a two percent gelatin sedimentation technique, as previously described (Parkos et al., J. Cell Biol. 117:757-764, 1992). Residual erythrocytes were thaen removed by gentle lysis in cold NH₄Cl lysis buffer. This technique allows for rapid isolation (90 minutes) of functionally active polymorphonuclear leukocytes (>98% as detected by trypan blue exclusion) at greater than 90% purity. Polymorphonuclear leukocytes were subsequently suspended in modified HBSS(−) (without Ca²⁺ and Mg²⁺, with 10 mM HEPES, pH 7.4, Sigma Chem. Co.) at a concentration of 5×10⁷/ml.

Before addition of polymorphonuclear leukocytes in this assay system, inverted cell culture inserts (Parkos et al., J. Cell Biol. 117:757-764, 1992) were extensively rinsed in HBSS(+) to remove residual serum components. S. enterica were prepared by washing twice in HBSS(+) and resuspended at a final concentration of approximately 5×10⁹/ml. Inverted cell culture inserts were removed from each well and placed in a moist chamber such that the epithelial apical membrane was oriented upward. The bacterial suspension (25 μl aliquots containing approximately 1.25×10⁸ CFU) was gently distributed onto the apical surface and incubated for sixty minutes at 37° C. Non-adherent bacteria were removed by washing three times in HBSS(+) buffer. The inverted cell culture inserts were then transferred back into the 24-well tissue culture tray containing 1.0 ml HBSS buffer in the lower (apical compartment) reservoir and 160 μl in the upper (basolateral compartment) reservoir) (McCormick et al., J. Cell Biol. 123:895-907, 1993). For simplicity, the reservoir will be referred to according to which epithelial membrane domain they interface with (i.e. apical or basolateral). To the basolateral bath, 40 μl (1×10⁶) of isolated polymorphonuclear leukocytes were added to each cell culture insert and incubated for 110 minutes at 37° C. Positive control transmigration assays were performed by addition of chemoattractant (1 μM fMLP) to the opposing apical reservoir. All the experiments were performed in a 37° C. room to ensure that epithelial monolayers, solutions, and plasticware were maintained at uniform 37° C. temperature (McCormick et al., J. Cell Biol. 123:895-907, 1993).

Transmigration was quantified by assaying for the polymorphonuclear leukocytes azurophilic granule marker myeloperoxidase as previously described (Parkos et al., J. Cell Biol. 117:757-764, 1992; Parkos et al., J. Clin. Invest. 88:1605-1612, 1991).

Typhoid Mouse Assay

Bacterial suspensions were grown overnight at 37° C. in LB broth with shaking. Next day, the bacteria were resuspended in phosphate-buffered saline. 4- to 6-week-old female BALB/c mice (10 mice per mutant) were inoculated orally with 100 μl of bacterial suspensions (10⁷ CFU/mouse), after being deprived of food for four hours. Mice survival was analyzed using the PRISM (version 2.00) computer program. The mouse protocol was reviewed and approved by the Animal Care Committee at Massachusetts General Hospital.

Isolation of Additional Virulence Genes

Based on the nucleotide and amino acid sequences described herein, the isolation of additional coding sequences of pathogenicity factors is made possible using standard strategies and techniques that are well known in the art. Any pathogenic cell can serve as the nucleic acid source for the molecular cloning of such a pathogenicity gene, and these sequences are identified as ones encoding a protein exhibiting pathogenicity-associated structures, properties, or activities.

In one particular example of such an isolation technique, any one of the nucleotide sequences described herein may be used, together with conventional screening methods of nucleic acid hybridization screening. Such hybridization techniques and screening procedures are well known to those skilled in the art and are described, for example, in Benton and Davis (Science 196:180-182, 1977); Grunstein and Hogness (Proc. Natl. Acad. Sci., USA 72:3961-3965, 1975); Ausubel et al. (Current Protocols in Molecular Biology, Wiley Interscience, New York, 1997); Berger and Kimmel (supra); and Sambrook et al., Molecular Cloning: A Laboratory Manual, Cold Spring Harbor Laboratory Press, New York. In one particular example, all or part of the srfH, spi4-F, rhuM, gmHA, leuO, rfaL, pipA, and cstA sequence (described herein) may be used as a probe to screen a recombinant bacterial DNA library for genes having sequence identity to the srfH, spi4-F, rhuM, gmHA, leuO, rfaL, pipA, and cstA genes. Hybridizing sequences are detected by plaque or colony hybridization according to standard methods.

Alternatively, using all or a portion of the amino acid sequence of the SrfH, Spi4-F, RhuM, GmHA, LeuO, RfaL, PipA, and CstA polypeptides, one may readily design srfH, spi4-F, rhuM, gmHA, leuO, rfaL, pipA, and cstA-specific oligonucleotide probes, including degenerate oligonucleotide probes (i.e., a mixture of all possible coding sequences for a given amino acid sequence). These oligonucleotides may be based upon the sequence of either DNA strand and any appropriate portion of the srfH (SEQ ID NO:1), rhuM (SEQ ID NO:3), spi4-F (SEQ ID NO:5), gmHA (SEQ ID NO:7), leuO (SEQ ID NO:9), rfaL (SEQ ID NO:11), cstA (SEQ ID NO:13) and pipA (SEQ ID NO:17) of the SrfH, Spi4-F, RhuM, GmHA, LeuO, RfaL, and CstA proteins. General methods for designing and preparing such probes are provided, for example, in Ausubel et al. (supra), and Berger and Kimmel, Guide to Molecular Cloning Techniques, 1987, Academic Press, New York. These oligonucleotides are useful for srfH, spi4-F, rhuM, gmHA, leuO, rfaL, and cstA gene isolation, either through their use as probes capable of hybridizing to srfH, spi4-F, rhuM, gmHA, leuO, rfaL, pipA, and cstA complementary sequences or as primers for various amplification techniques, for example, polymerase chain reaction (PCR) cloning strategies. If desired, a combination of different, detectably-labelled oligonucleotide probes may be used for the screening of a recombinant DNA library. Such libraries are prepared according to methods well known in the art, for example, as described in Ausubel et al. (supra), or they may be obtained from commercial sources.

As discussed above, sequence-specific oligonucleotides may also be used as primers in amplification cloning strategies, for example, using PCR. PCR methods are well known in the art and are described, for example, in PCR Technology, Erlich, ed., Stockton Press, London, 1989; PCR Protocols: A Guide to Methods and Applications, Innis et al., eds., Academic Press, Inc., New York, 1990; and Ausubel et al. (supra). Primers are optionally designed to allow cloning of the amplified product into a suitable vector, for example, by including appropriate restriction sites at the 5′ and 3′ ends of the amplified fragment (as described herein). If desired, nucleotide sequences may be isolated using the PCR “RACE” technique, or Rapid Amplification of cDNA Ends (see, e.g., Innis et al. (supra)). By this method, oligonucleotide primers based on a desired sequence are oriented in the 3′ and 5′ directions and are used to generate overlapping PCR fragments. These overlapping 3′- and 5′-end RACE products are combined to produce an intact fill-length cDNA. This method is described in Innis et al. (supra); and Frohman et al., Proc. Natl. Acad. Sci. USA 85:8998, 1988.

Partial virulence sequences, e.g., sequence tags, are also useful as hybridization probes for identifying full-length sequences, as well as for screening databases for identifying previously unidentified related virulence genes. Confirmation of a sequence's relatedness to a pathogenicity polypeptide may be accomplished by a variety of conventional methods including, but not limited to, functional complementation assays and sequence comparison of the gene and its expressed product. In addition, the activity of the gene product may be evaluated according to any of the techniques described herein, for example, the functional or immunological properties of its encoded product.

Once an appropriate sequence is identified, it is cloned according to standard methods and may be used, for example, for screening compounds that reduce the virulence of a pathogen.

Polypeptide Expression

In general, polypeptides of the invention may be produced by transformation of a suitable host cell with all or part of a polypeptide-encoding nucleic acid molecule or fragment thereof in a suitable expression vehicle.

Those skilled in the field of molecular biology will understand that any of a wide variety of expression systems may be used to provide the recombinant protein. The precise host cell used is not critical to the invention. A polypeptide of the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exists for the production of the polypeptides of the invention. Such vectors include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains which express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system which is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Once the recombinant polypeptide of the invention is expressed, it is isolated, e.g., using affinity chromatography. In one example, an antibody (e.g., produced as described herein) raised against a polypetide of the invention may be attached to a column and used to isolate the recombinant polypeptide. Lysis and fractionation of polypeptide-harboring cells prior to affinity chromatography may be performed by standard methods (see, e.g., Ausubel et al., supra).

Once isolated, the recombinant protein can, if desired, be further purified, e.g., by high performance liquid chromatography (see, e.g., Fisher, Laboratory Techniques In Biochemistry And Molecular Biology, eds., Work and Burdon, Elsevier, 1980).

Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.).

These general techniques of polypeptide expression and purification can also be used to produce and isolate useful peptide fragments or analogs (described herein).

Antibodies

To generate antibodies, a coding sequence for a polypeptide of the invention may be expressed as a C-terminal fusion with glutathione S-transferase (GST) (Smith et al. Gene 67:31-40, 1988). The fusion protein is purified on glutathione-Sepharose beads, eluted with glutathione, cleaved with thrombin (at the engineered cleavage site), and purified to the degree necessary for immunization of rabbits. Primary immunizations are carried out with Freund's complete adjuvant and subsequent immunizations with Freund's incomplete adjuvant. Antibody titres are monitored by Western blot and immunoprecipitation analyses using the thrombin-cleaved protein fragment of the GST fusion protein. Immune sera are affinity purified using CNBr-Sepharose-coupled protein. Antiserum specificity is determined using a panel of unrelated GST proteins.

As an alternate or adjunct immunogen to GST fusion proteins, peptides corresponding to relatively unique immunogenic regions of a polypeptide of the invention may be generated and coupled to keyhole limpet hemocyanin (KLH) through an introduced C-terminal lysine. Antiserum to each of these peptides is similarly affinity purified on peptides conjugated to BSA, and specificity tested in ELISA and Western blots using peptide conjugates, and by Western blot and immunoprecipitation using the polypeptide expressed as a GST fusion protein.

Alternatively, monoclonal antibodies which specifically bind any one of the polypeptides of the invention are prepared according to standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., In Monoclonal Antibodies and T Cell Hybridomas, Elsevier, N.Y., 1981; Ausubel et al., supra). Once produced, monoclonal antibodies are also tested for specific recognition by Western blot or immunoprecipitation analysis (by the methods described in Ausubel et al., supra). Antibodies which specifically recognize the polypeptide of the invention are considered to be useful in the invention; such antibodies may be used, e.g., in an immunoassay. Alternatively monoclonal antibodies may be prepared using the polypeptide of the invention described above and a phage display library (Vaughan et al. Nature Biotech 14:309-314, 1996).

Preferably, antibodies of the invention are produced using fragments of the polypeptide of the invention which lie outside generally conserved regions and appear likely to be antigenic, by criteria such as high frequency of charged residues. In one specific example, such fragments are generated by standard techniques of PCR and cloned into the pGEX expression vector (Ausubel et al., supra). Fusion proteins are expressed in E. coli and purified using a glutathione agarose affinity matrix as described in Ausubel et al. (supra). To attempt to minimize the potential problems of low affinity or specificity of antisera, two or three such fusions are generated for each protein, and each fusion is injected into at least two rabbits. Antisera are raised by injections in a series, preferably including at least three booster injections.

Screening Assays for Host Pathogen Defense Response Genes

To identify host pathogen defense response genes, mutagenized nematodes are identified for decreased levels of Salmonella-induced gonadal programmed cell death. In one approach, young adult mutagenized nematodes are transferred to plates containing Salmonella bacterial lawns. The number of corpses present in the gonad of a mutagenized nematode is measured using any standard method (e.g., microscopy) and compared to the gonadal programmed cell death that occurs in a wild-type control nematode. Mutagenized worms having reduced gonadal cell death are selected, and the mutation is used to identify a host pathogen defense response gene using standard methods (e.g., mapping and cloning).

Additional components of the host pathogen defense response pathway are identified using standard nematode genetic screens for suppressors of the cell death defective phenotype (e.g., ced-9 loss of function). Nematode survival is assayed on a pathogen (e.g., Salmonella). Those worms identified as having increased survival on the pathogen are useful for identifying a host pathogen response gene.

Other components of the host pathogen response pathway are identified using standard genetic screens for suppressors of increased gonadal programmed cell death phenotype or hypersusceptibility to pathogen-induced killing phenotype.

Screening Assays for Microbial Effector Proteins

As is discussed above, our experimental results demonstrated that expression of a Salmonella effector protein arrested nematode embryo development. Based on this discovery, we have developed a screening procedure for identifying microbial effector proteins in vivo. In general, the method includes introducing a candidate microbial gene encoding an effector protein into a nematode and determining whether expression of the gene under an appropriate control element (e.g., a conditional or constitutive promoter) alters a phenotype of the nematode as compared to a control nematode (e.g., a wild-type nematode). Phenotypes assayed using this method include enhanced susceptibility to pathogen, embryonic development, lifespan, locomotion, touch sensitivity, cell death (e.g., programmed cell death or gonadal programmed cell death), or cell proliferation. Assays for alterations in such phenotypes are standard in the art and are described by Riddle et al. (C. elegans II, Plainview (N.Y.), Cold Spring Harbor Laboratory Press, 1997).

Based on our results demonstrating that expression of a microbial effector protein in a nematode alters its phenotype, it will be readily understood that a compound that inhibits the pathogenicity of an effector protein may also be identified. For example, a nematode, expressing an effector protein (e.g., SrfH) as described above is cultured with a test compound according to standard methods. A compound that rescues (or suppresses or reduces) the altered nematode phenotype (e.g., embryonic arrest) is taken as being useful in the invention.

Screening Assays for Therapeutics

As discussed above, we have identified a number of Salmonella enterica virulence factors that are involved in pathogenicity and that may therefore be used to screen for compounds that reduce the virulence of that organism, as well as other microbial pathogens. Any number of methods are available for carrying out such screening assays. According to one approach, candidate compounds are added at varying concentrations to the culture medium of pathogenic cells expressing one of the nucleic acid sequences of the invention. Gene expression is then measured, for example, by standard Northern blot analysis (Ausubel et al., supra), using any appropriate fragment prepared from the nucleic acid molecule as a hybridization probe. The level of gene expression in the presence of the candidate compound is compared to the level measured in a control culture medium lacking the candidate molecule. A compound which promotes a decrease in the expression of the pathogenicity factor is considered useful in the invention; such a molecule may be used, for example, as a therapeutic to combat the pathogenicity of an infectious organism.

If desired, the effect of candidate compounds may, in the alternative, be measured at the level of polypeptide production using the same general approach and standard immunological techniques, such as Western blotting or immunoprecipitation with an antibody specific for a pathogenicity factor. For example, immunoassays may be used to detect or monitor the expression of at least one of the polypeptides of the invention in a pathogenic organism. Polyclonal or monoclonal antibodies (produced as described above) which are capable of binding to such a polypeptide may be used in any standard immunoassay format (e.g., ELISA, Western blot, or RIA assay) to measure the level of the pathogenicity polypeptide. A compound which promotes a decrease in the expression of the pathogenicity polypeptide is considered particularly useful. Again, such a molecule may be used, for example, as a therapeutic to combat the pathogenicity of an infectious organism.

Alternatively, or in addition, candidate compounds may be screened for those which specifically bind to and inhibit a pathogenicity polypeptide of the invention. The efficacy of such a candidate compound is dependent upon its ability to interact with the pathogenicity polypeptide. Such an interaction can be readily assayed using any number of standard binding techniques and functional assays (e.g., those described in Ausubel et al, supra). For example, a candidate compound may be tested in vitro for interaction and binding with a polypeptide of the invention (for example, any of those polypeptides described herein) and its ability to modulate pathogenicity may be assayed by any standard assays (e.g., those described herein).

In one particular example, a candidate compound that binds to a pathogenicity polypeptide may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the pathogenicity polypeptide is identified on the basis of its ability to bind to the pathogenicity polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). In addition, these candidate compounds may be tested for their ability to render a pathogen less virulent (e.g., as described herein). Compounds isolated by this approach may also be used, for example, as therapeutics to treat or prevent the onset of a pathogenic infection, disease, or both. Compounds which are identified as binding to pathogenicity polypeptides with an affinity constant less than or equal to 10 mM are considered particularly useful in the invention.

In yet another approach, candidate compounds are screened for the ability to inhibit the virulence of a Salmonella enterica cell by monitoring the effect of the compound on the production of SrfH, Spi4-F, RhuM, GmHA, LeuO, RfaL, PipA, and CstA. According to one approach, candidate compounds are added at varying concentrations to a culture medium of pathogenic cells. SrfH, Spi4-F, RhuM, GmHA, LeuO, RfaL, PipA, and CstA is then measured according to any standard method. The level of SrfH, Spi4-F, RhuM, GmHA, LeuO, RfaL, PipA, and CstA production in the presence of the candidate compound is compared to the level measured in a control culture lacking the candidate molecule. A compound, for example, which promotes a decrease in the expression of a SrfH, Spi4-F, RhuM, GmHA, LeuO, RfaL, PipA, and CstA is considered useful in the invention; such a molecule may be used, for example, as a therapeutic to combat the enteritis caused by the Salmonella. Optionally, compounds identified in any of the above-described assays may be confirmed as useful in conferring protection against the development of a pathogenic infection in any standard animal model (e.g., the mouse-burn assay described herein) and, if successful, may be used as anti-pathogen therapeutics.

Test Compounds and Extracts

In general, compounds capable of reducing pathogenic virulence are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-pathogenic activity should be employed whenever possible.

When a crude extract is found to have an anti-pathogenic or anti-virulence activity, or a binding activity, further fractionation of the positive lead extract is necessary to isolate chemical constituents responsible for the observed effect. Thus, the goal of the extraction, fractionation, and purification process is the careful characterization and identification of a chemical entity within the crude extract having anti-pathogenic activity. Methods of fractionation and purification of such heterogenous extracts are known in the art. If desired, compounds shown to be useful agents for the treatment of pathogenicity are chemically modified according to methods known in the art.

Vaccine Production

The invention also provides for a method of inducing an immunological response in an individual, particularly a human, which comprises inoculating the individual with the polypeptides of the invention or fragments thereof, in a suitable carrier for the purpose of inducing an immune response to protect an individual from infection, particularly bacterial infection, and most particularly Salmonella infection. The administration of this immunological composition may be used either therapeutically in individuals already experiencing bacterial infection, or may be used prophylactically to prevent bacterial infection.

The preparation of vaccines that contain immunogenic polypeptides is known to one skilled in the art. The polypeptide may serve as an antigen for vaccination, or an expression vector encoding the polypeptide, or fragments or variants thereof, might be delivered in vivo in order to induce an immunological response comprising the production of antibodies or a T cell immune response.

For example, the SrfH, Spi4-F, RhuM, GmHA, LeuO, RfaL, PipA, and CstA polypeptides or fragments or variants thereof might be delivered in vivo in order to induce an immune response. The polypeptides might be fused to a recombinant protein that stabilizes the polypeptide of the invention, aids in its solubilization, facilitates its production or purification, or acts as an adjuvant by providing additional stimulation of the immune system. The compositions and methods comprising the polypeptides or nucleotides of the invention and immunostimulatory DNA sequences are described in (Sato, et al., Science 273: 352 (1996)).

Typically vaccines are prepared in an injectable form, either as a liquid solution or as a suspension. Solid forms suitable for injection may also be prepared as emulsions, or with the polypeptides encapsulated in liposomes. Vaccine antigens are usually combined with a pharmaceutically acceptable carrier, which includes any carrier that does not induce the production of antibodies harmful to the individual receiving the carrier. Suitable carriers typically comprise large macromolecules that are slowly metabolized, such as proteins, polysaccharides, polylactic acids, polyglycolic acids, polymeric amino acids, amino acid copolymers, lipid aggregates, and inactive virus particles. Such carriers are well known to those skilled in the art. These carriers may also function as adjuvants.

Adjuvants are immunostimulating agents that enhance vaccine effectiveness. Effective adjuvants include, but are not limited to, aluminum salts such as aluminum hydroxide and aluminum phosphate, muramyl peptides, bacterial cell wall components, saponin adjuvants, and other substances that act as immunostimulating agents to enhance the effectiveness of the composition.

Immunogenic compositions, i.e. the antigen, pharmaceutically acceptable carrier and adjuvant, also typically contain diluents, such as water, saline, glycerol, ethanol. Auxiliary substances may also be present, such as wetting or emulsifying agents, pH buffering substances, and the like. Proteins may be formulated into the vaccine as neutral or salt forms. The vaccines are typically administered parenterally, by injection; such injection may be either subcutaneously or intramuscularly. Additional formulations are suitable for other forms of administration, such as by suppository or orally. Oral compositions may be administered as a solution, suspension, tablet, pill, capsule, or sustained release formulation.

In addition, the vaccine can also be administered to individuals to generate polyclonal antibodies (purified or isolated from serum using standard methods) that may be used to passively immunize an individual. These polyclonal antibodies can also serve as immunochemical reagents.

It is also possible to prepare live attenuated microorganism vaccines that express recombinant polypeptides, for example, of the SrfH, Spi4-F, RhuM, GmHA, LeuO, RfaL, PipA, and CstA antigens. Suitable attenuated microorganisms are known in the art, and include, for example, viruses and bacteria.

Vaccines are administered in a manner compatible with the dose formulation. The immunogenic composition of the vaccine comprises an immunologically effective amount of the antigenic polypeptides and other previously mentioned components. By an immunologically effective amount is meant a single dose, or a vaccine administered in a multiple dose schedule, that is effective for the treatment or prevention of an infection. The dose administered will vary, depending on the subject to be treated, the subject's health and physical condition, the capacity of the subject's immune system to produce antibodies, the degree of protection desired, and other relevant factors. Precise amounts of the active ingredient required will depend on the judgement of the practitioner, but typically range between 5 μg to 250 μg of antigen per dose.

Use

The methods of the invention provide a simple means for identifying bacterial virulence factors (such as Salmonella virulence factors) and compounds capable of either inhibiting pathogenicity or enhancing an organism's resistance capabilities to such pathogens. Accordingly, a chemical entity discovered to have medicinal value using the methods described herein are useful as either drugs, or as information for structural modification of existing anti-pathogenic compounds, e.g., by rational drug design.

For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections which provide continuous, sustained levels of the drug in the patient. Treatment of human patients or other animals will be carried out using a therapeutically effective amount of an anti-pathogenic agent in a physiologically-acceptable carrier. In the context of treating a bacterial infection a “therapeutically effective amount” or “pharmaceutically effective amount” indicates an amount of an antibacterial agent, e.g., as disclosed for this invention, which has a therapeutic effect. This generally refers to the inhibition, to some extent, of the normal cellular functioning of bacterial cells (e.g., Salmonella cells) causing or contributing to a bacterial infection. The dose of antibacterial agent which is useful as a treatment is a “therapeutically effective amount.” Thus, as used herein, a therapeutically effective amount means an amount of an antibacterial agent which produces the desired therapeutic effect as judged by clinical trial results, standard animal models of infection, or both. This amount can be routinely determined by one skilled in the art and will vary depending upon several factors, such as the particular bacterial strain involved and the particular antibacterial agent used. This amount can further depend on the patient's height, weight, sex, age, and renal and liver function or other medical history. For these purposes, a therapeutic effect is one which relieves to some extent one or more of the symptoms of the infection and includes curing an infection.

The compositions containing antibacterial agents of virulence factors or genes can be administered for prophylactic or therapeutic treatments, or both. In therapeutic applications, the compositions are administered to a patient already suffering from an infection from bacteria (similarly for infections by other microbes), in an amount sufficient to cure or at least partially arrest the symptoms of the infection. An amount adequate to accomplish this is defined as “therapeutically effective amount.” Amounts effective for this use will depend on the severity and course of the infection, previous therapy, the patient's health status and response to the drugs, and the judgment of the treating physician. In prophylactic applications, compositions containing the compounds of the invention are administered to a patient susceptible to, or otherwise at risk of, a particular infection. Such an amount is defined to be a “prophylactically effective amount.” In this use, the precise amounts again depend on the patient's state of health, weight, and the like. However, generally, a suitable effective dose will be in the range of 0.1 to 10000 milligrams (mg) per recipient per day, preferably in the range of 10-5000 mg per day. The desired dosage is preferably presented in one, two, three, four, or more subdoses administered at appropriate intervals throughout the day. These subdoses can be administered as unit dosage forms, for example, containing 5 to 1000 mg, preferably 10 to 100 mg of active ingredient per unit dosage form. Preferably, the compounds of the invention will be administered in amounts of between about 2.0 mg/kg to 25 mg/kg of patient body weight, between about one to four times per day.

Suitable carriers and their formulation are described, for example, in Remington's Pharmaceutical Sciences by E. W. Martin. The amount of the anti-pathogenic agent to be administered varies depending upon the manner of administration, the age and body weight of the patient, and with the type of disease and extensiveness of the disease. Generally, amounts will be in the range of those used for other agents used in the treatment of other microbial diseases, although in certain instances lower amounts will be needed because of the increased specificity of the compound. A compound is administered at a dosage that inhibits microbial proliferation.

All publications and patents mentioned in this specification are herein incorporated by reference to the same extent as if each individual publication or patent was specifically and individually indicated to be incorporated by reference.

While the invention has been described in connection with specific embodiments thereof, it will be understood that it is capable of further modifications. This application is intended to cover any variations, uses, or adaptations following, in general, the principles of the invention and including such departures from the present disclosure within known or customary practice within the art to which the invention pertains and may be applied to the essential features hereinbefore set forth.

What is claimed is:

1. A substantially pure polypeptide comprising an amino acid sequence having substantial sequence identity to the amino acid sequence of GmhA (SEQ ID NO:8).

2. The substantially pure polypeptide of claim 1, said polypeptide comprising the amino acid sequence of GmhA (SEQ ID NO:8).

3. The substantially pure polypeptide of claim 2, wherein said amino acid sequence consists essentially of the amino acid sequence of GmhA (SEQ ID NO:8) or a fragment thereof.

4. A substantially pure polypeptide fragment of the substantially pure polypeptide of claim 1.

5. The substantially pure polypeptide fragment of claim 4, wherein said polypeptide fragment comprises 200 contiguous amino acids of SEQ ID NO:8.

6. An isolated nucleic acid molecule having substantial identity to the nucleotide sequence of gmhA (SEQ ID NO:7).

7. The isolated nucleic acid molecule of claim 6, said nucleic acid molecule comprising the nucleotide sequence of gmhA (SEQ ID NO:7) or a complement thereof.

8. The isolated nucleic acid molecule of claim 7, said nucleic acid molecule consisting essentially of the nucleotide sequence of gmhA (SEQ ID NO:7) or a fragment thereof.

9. A vector comprising the isolated nucleic acid molecule of any one of claim 6.

10. A host cell comprising the vector of claim 9.

11. A screening method for identifying a compound that modulates gene expression of a nucleic acid molecule in a microorganism, said method comprising the steps of:
(a) providing a microbial cell comprising a nucleic acid molecule having substantial identity to the nucleotide sequence of gmhA (SEQ ID NO:7);
(b) contacting said microbial cell with a compound; and
(c) comparing the level of gene expression of said nucleic acid molecule in the presence of said compound with the level of gene expression in the absence of said compound; wherein a measurable difference in gene expression indicates that said compound modulates gene expression of said nucleic acid molecule in a microorganism.

12. The method of claim 11, wherein said screening method identifies a compound that increases transcription of said nucleic acid molecule.

13. The method of claim 11, wherein said screening method identifies a compound that decreases transcription of said nucleic acid molecule.

14. The method of claim 11, wherein said screening method identifies a compound that increases translation of an mRNA transcribed from said nucleic acid molecule.

15. The method of claim 11, wherein said screening method identifies a compound that decreases translation of an mRNA transcribed from said nucleic acid molecule.

16. The method of claim 11, wherein the compound is a member of a chemical library.

17. The method of claim 11, wherein said microbial cell belongs to the genus Salmonella.

18. The method of claim 11, wherein the activity of the compound is dependent upon the presence of the gmhA gene (SEQ ID NO:7) or a functional equivalent thereof.

19. The method of claim 11, wherein said compound targets the gmhA gene (SEQ ID NO:7) or a functional equivalent thereof.

20. A screening method for identifying a compound that modulates an activity of a polypeptide in a microorganism, said method comprising the steps of:
(a) providing a microbial cell expressing a polypeptide having substantial identity to the amino acid sequence of GmhA (SEQ ID NO:8);
(b) contacting said microbial cell with a compound; and
(c) comparing an activity of said polypeptide in the presence of said compound with said activity in the absence of said compound; wherein a measurable difference in the activity indicates that said compound modulates said activity of said polypeptide.

21. The method of claim 20, wherein said screening method identifies a compound that increases the activity of said polypeptide.

22. The method of claim 20, wherein said screening method identifies a compound that decreases the activity of said polypeptide.

23. The method of claim 20, wherein the compound is a member of a chemical library.

24. The method of claim 20, wherein comparing the activity of the polypeptide involves an immunological assay.

25. The method of claim 20, wherein said microbial cell belongs to the genus Salmonella.

26. The method of claim 20, wherein said polypeptide is the substantially pure polypeptide of claim 1.

27. A method for identifying a compound which binds a polypeptide, said method comprising the steps of: (a) contacting a test compound with a substantially pure polypeptide comprising an amino acid sequence having substantial sequence identity to the amino acid sequence of GmhA (SEQ ID NO:8) under conditions that allow binding; and (b) detecting binding of the test compound to the polypeptide.

28. The method of claim 27, wherein said polypeptide comprises the amino acid sequence of GmhA (SEQ ID NO:8).

29. The method of claim 27, wherein the amino acid sequence of GmhA consists essentially of the amino acid sequence of SEQ ID NO:8 or a fragment thereof.

30. The method of claim 11, wherein the compound is a member of a chemical library.

31. A method of identifying a compound that inhibits the pathogenicity of an effector protein in a nematode, said method comprising:
(a) providing a nematode expressing an effector protein;
(b) contacting said nematode with a test compound; and
(c) determining whether said test compound inhibits the pathogenicity of said polypeptide in the nematode.

32. The method of claim 31, wherein said effector protein is a microbial effector protein.

33. The method of claim 32, wherein said microbial effector protein is a Salmonella effector protein.

34. The method of claim 33, wherein said effector protein is SrfH (SEQ ID NO: 2).

35. The method of claim 31, wherein said effector protein is encoded by an isolated nucleic acid molecule having a nucleic acid sequence that is substantially identical to the srfH nucleic acid sequence shown in SEQ ID NO:1.

36. The method of claim 31, wherein said test compound is provided in a compound library.

37. The method of claim 31, wherein said nematode is an adult nematode or is a nematode embryo.
